(19) United States
(12) Patent Application Publication
(10) Pub. No.: US 2004/0259142 A1
(43) Pub. Date: Dec. 23, 2004

(54) Products and methods

(75) Inventors: Wengang Chai, Harrow (GB); Ten Feizi, Harrow (GB); Alexander Lawson, Harrow (GB)

Correspondence Address: NIKOLAI & MERSEREAU, P.A., 900 SECOND AVENUE SOUTH, SUITE 820, MINNEAPOLIS, MN, 55402, US

(73) Assignee: Imperial College Innovations Limited, London (GB)

(21) Appl. No.: 10/855,072

(22) Filed: May 27, 2004

Related U.S. Application Data
(60) Provisional application No. 60/475,625, filed on Jun. 4, 2003.

(51) Int. Cl. C12Q 1/68; G01N 33/53; C12N 9/10; C12M 1/34

(52) U.S. Cl. 435/6; 435/7.1; 435/287.2

(57) ABSTRACT

An array of reducing end-tagged carbohydrate molecules immobilised on a support. The reducing end-tagged carbohydrate molecules may be neoglycolipids or glycolipids. The reducing end-tagged carbohydrate molecule may have a carbohydrate which is an oligosaccharide, monosaccharide, an N-glycan, or an O-glycan. The array may comprise one or more samples of reducing end-tagged carbohydrate molecules which comprise a homogeneous sample of carbohydrate or a heterogeneous sample of carbohydrate. The carbohydrate may be derived from a microbe, a specific cell type, a specific tissue or organ, an animal, for example a human, or a plant.

A method of determining whether a molecule interacts with a carbohydrate comprising: i) contacting an array according to the invention with the molecule; and ii) measuring whether the molecule binds to any of the arrayed reducing end-tagged carbohydrate molecule.

The present invention relates to arrays of carbohydrate molecules immobilised on a support, and methods using such arrays.

Carbohydrate chains are prominent components at the surface of mammalian cells occurring as N- and O-glycans on glycoproteins, glycosaminoglycan chains on proteoglycans, and oligosaccharides of glycolipids. Carbohydrate chains also occur on many secreted and extracellular glycoproteins. They range in length from two to more than two hundred monosaccharide residues, and they are almost unfathomably diverse. The term glycome has been coined for the repertoire of oligosaccharide structures in an organism.

Cellular glycoconjugates play important roles in many biological processes, including events of molecular recognition at fertilisation (Focarelli et al (2001) Cells Tiss Org 168, 76-81; Rosati et al (2000) Int J Dev Biol 44, 609-618) and processes of cell-cell recognition, adhesion, and cell activation throughout the development and maturation of a living organism (Feizi (1982) Adv Exp Med Biol 152, 167-177; Crocker and Feizi (1996) Curr Opin Struc Biol 6, 679-691; Feizi (2000) Glycoconj J 17, 553-565; Feizi (2000) Immunol Rev 173, 79-88). Abnormalities in the expression of complex carbohydrates are found in cancer (Hakomori (1985) Cancer Res 45, 2405-2414; Sell (1990) Hum Pathol 21, 1003-1019) retrovirus infection (Adachi et al (1988) J Exp Med 167, 323-331; Nakaishi et al (1988) Cancer Res 48, 1753-1758) and other diseases (Schachter and Jaeken (1999) Biochem Biophys Acta 1455, 179-192. Carbohydrate structures also play critical roles in host-microorganism interactions. Many attachment sites on host cells (receptors or co-receptors) for microbes are glycoconjugates (Karlsson et al (1992) APMIS Suppl 27, 71-83); their structural diversity and selectivity of host tissue expression contribute significantly to the tropism of microbial infections (Karlsson et al (1992) APMIS Suppl 27, 71-83; Feizi and Loveless (1996) Am J Respir Crit Care Med 154, S133-136).

Post genome, with the realization that the number of proteins encoded in the human genome is fewer than anticipated, there is heightened interest in carbohydrates and ways in which they diversify proteins and may modulate their activities and functions in health and disease. One challenge, for example, is to determine the repertoire of carbohydrate-binding proteins. There are an increasing number of receptors known to operate through binding to specific oligosaccharides. Among them are proteins that mediate critical processes such as protein folding and trafficking, and play key roles in cell-mediated and humoral mechanisms of inflammation and immunity (Helenius and Aebi (2001) Science 291, 2364-2369; Feizi (2000) Immunol Rev 173, 79-88; Crocker and Varki (2001) Trends Immunol 22, 337-342; Weis et al (1998) Immunol Rev 163, 19-34). Moreover, as mentioned above, a considerable number of pathogens have evolved to produce adhesive proteins that bind to specific carbohydrate sequences on host cells at the initial stages of infection (Karlsson (1998) Mol Microbiol 29, 1-11). This knowledge has served to stimulate ideas on carbohydrate-based therapeutics.

Developments in assignments of roles for carbohydrate sequences as ligands have lagged behind those for nucleic acid and protein sequences. This is in part because of the remarkable heterogeneities of carbohydrates, and the relatively small amounts that can be isolated. There are two other major factors. The first is that oligosaccharide ligands cannot be readily cloned, being products of multiple glycosyltransferases. Second, the affinities of most carbohydrate-protein interactions are so low that di- or multivalence both of oligosaccharide and of the recognition protein is required for sensitive detection in binding experiments such as precipitation and radioimmunoassays, or ELISA-type experiments. Isolated free oligosaccharides can be examined only as inhibitors of such interactions. Hence there is a need for simple and readily accessible high-throughput analysis techniques to be developed.

Microarrays have been reported to be one of the most frequently used approaches because large libraries of compounds can be quickly screened and only small quantities of material are required (Fodor et al (1991) Science 251, 767-773; Whitesides and Love (2001) Sci Am 285, 38-47; Cui et al (2001) Science 293, 1289-1292), an important consideration due to the low availability of some complex carbohydrates. Few approaches have been developed, thus far, for the fabrication of carbohydrate microarrays.

Wang et al (2002) Nature Biotech 20, 275-281 report that nitrocellulose-coated glass slides can be used to immobilise microspots of carbohydrate polymers without covalent conjugation.

Houseman and Mrksich (2002) Chemistry Biology 9, 443-454 report that carbohydrate arrays can be prepared by the Diels-Alder mediated immobilisation of carbohydrate-cyclopentadiene conjugates to a gold surface.

The present invention provides an array of reducing end-tagged carbohydrate molecules immobilised on a support.

By ‘array’ we mean that one or more reducing end-tagged carbohydrate molecules are immobilised as an organised arrangement or pattern at two or more locations on the support. The type of sample at each location is known. The term ‘array’ will be well known to those skilled in the art. See, for example, EP 0 804 731. Microarrays are also considered to be an embodiment of the first aspect of the invention. A microarray may typically have sample locations separated by a distance of 50-200 microns or less and immobilised sample in the nano to micromolar range or nano to picogram range. See, for example, EP 0 804 731.

Typically, an array will have at least 4, 8, 16, 24, 48, 96 or several hundred or thousand sample locations.

Arrays of other biomolecules are well known in the art, for example protein and nucleic acid arrays.

Biological chips or arrays have immobilised molecules arranged in arrays, with each immobilised molecule assigned a specific location. Biological chips have been produced in which each location has a scale of, for example, ten microns. The chips can be used to determine whether target molecules interact with any of the immobilised molecules on the chip. After exposing the array to target molecules under selected test conditions, scanning devices can examine each location in the array and determine whether a target molecule has interacted with the immobilised molecule or molecules at that location.

Biological chips or arrays are useful in a variety of screening techniques for obtaining information about either the immobilised molecules or the target molecules. For example, a library of immobilised reducing end-tagged carbohydrate molecules can be used in screening for drugs. Or the reducing end-tagged carbohydrate molecules can be exposed to a receptor, and those molecules that bind to the receptor can be identified. Molecules that interfere with such binding can be identified.

It is important to consider arrangements in which the reducing end-tagged carbohydrate molecules can be arrayed. There are least two possible variables: the composition of the reducing end-tagged carbohydrate molecules at each location of the array, and the composition of the reducing end-tagged carbohydrate molecules at different locations of the array.

Therefore, one array included in this embodiment of the invention is where the same reducing end-tagged carbohydrate molecules (for example as a complex sample) are immobilised on the support at one or more locations; each of these locations containing the same reducing end-tagged carbohydrate molecules or mixture of molecules. Hence this array includes an organised arrangement of samples of the same reducing end-tagged carbohydrate molecules (or same mixture of molecules).

The location may contain the same quantities of the reducing end-tagged carbohydrate molecules or mixture of molecules or may contain different quantities.

A further array included in this embodiment of the invention is where locations of the array differ in the reducing end-tagged carbohydrate molecules or mixture of molecules that they contain. Hence this array includes an organised arrangement of samples of different reducing end-tagged carbohydrate molecules or different mixtures of reducing end-tagged carbohydrate molecules.

For example, the array may include samples of complex mixtures of reducing end-tagged carbohydrate molecules from different cell types or tissues; or may include samples that correspond to fractions of such complex mixtures; or may include samples that correspond to single molecular species isolated from such fractions or prepared synthetically. Each type of sample may be present in more than one location (optionally at different concentrations) and one array may include examples of each such type of sample.

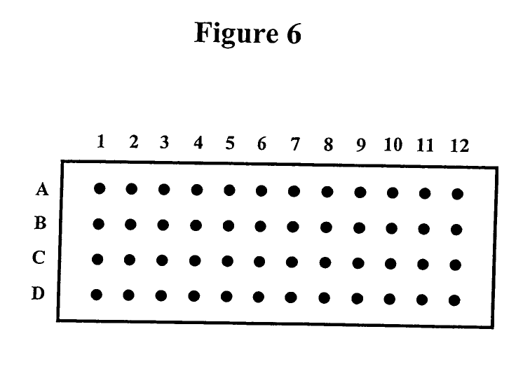

Non-limiting arrangements in which reducing end-tagged carbohydrate molecules may be arrayed are shown in FIG. 6. Here an array according to the first aspect of the invention is shown on which is immobilised reducing end-tagged carbohydrate molecules. The molecules present at location A1 may be identical to the molecules present at location A2, but A1 may have 10 times more molecules than at A2. Alternatively the molecules present at A1 may differ to those at A2, while B1 may be a repeat location to that of A1 for the purposes of a control, ie A1 and B1 contain the same molecules. Other arrangements will be appreciated by those skilled in the art.

As will be set out below, the reducing end-tagged carbohydrate molecules can be prepared from a broad range of sources. Therefore the arrays included in this aspect of the invention can have immobilised reducing end-tagged carbohydrate molecules or mixture of molecules prepared from the same source, or different sources.

By ‘reducing end-tagged carbohydrate molecules’ we mean carbohydrate conjugated at its reducing terminus to a molecule as a means of immobilising carbohydrates to a support. Example of such molecules include glycolipids and neoglycolipids.

Hence an embodiment of this aspect of the invention is where the reducing end-tagged carbohydrate molecules used are glycolipids. A further embodiment of this aspect of the invention is where the reducing end-tagged carbohydrate molecules used are neoglycolipids.

Neoglycolipids are monosaccharides or oligosaccharides chemically conjugated to lipid molecules (Tang et al (1985) Biochem Biophys Res Comm 132, 474-480; Feizi et al (1994) Methods Enzymol 230, 484-519). Such lipid molecules include phosphatidylethanolamine-type aminolipids.

Tagging of each monosaccharide or oligosaccharide with a hydrophobic lipid tail confers amphipathic property such that the neoglycolipids can be immobilized with the carbohydrates considered to be displayed in a cluster.

The lipid molecules which can be used as tags for neoglycolipids include dipalmitoyl phosphatidylethanolamine (DPPE), 1,2-dihexadecyl-sn-glycero-3-phosphoethanolamine (DHPE) and N-aminoacetyl-N-(9-anthracenylmethyl)-1,2-dihexadecyl-sn-glycero-3-phosphoethanolamine (ADHP). The tag may have a 10, 15, 18, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55 or 60 carbon atom length. It is preferred that the neoglycolipids used in this embodiment of the invention comprise a tag of between 24 to 50 carbon atom length.

The reducing end-tagged carbohydrate molecules may have a lipid tag of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35 or 40 carbon atom length. It is preferred that the reducing end-tagged carbohydrate molecules used in this aspect of the invention comprise a tag of between 5 to 25 carbon atom length, with an aliphatic or aromatic hydrocarbon backbone.

Also preferred is an embodiment of the first aspect of the invention where the tag or the resulting neoglycolipid has a chromophore. For example, aminopyridine has a chromophore.

An advantage of arraying reducing end-tagged carbohydrate molecules is that the carbohydrate portion of the molecule can be prepared from a broad range of sources. As a consequence of this, and in contrast to other carbohydrate arrays, the arrays of the first aspect of the invention can display carbohydrates prepared from a wide range of sources, as outlined further below.

Also, as the carbohydrates can be derived from natural sources, the range of carbohydrates arrayed is not limited by what can be artificially generated. Diverse glycomes comprising both known and unknown carbohydrates can be accessed for arraying

In addition, the carbohydrate portion of the arrayed molecules can be monosaccharides or oligosaccharides derived from larger, more complex carbohydrate containing molecules. Hence should a molecule interact with an arrayed reducing end-tagged carbohydrate molecule then the carbohydrate recognised is likely to have a more simple structure in relation to the full carbohydrate from which it was derived and thus be more readily defined.

In a further embodiment of this aspect of the invention the carbohydrate portion of the arrayed reducing end-tagged carbohydrate molecules is an oligosaccharide. By ‘oligosaccharide’ we mean a linear or branched chain of monosaccharides attached to one another via glycosidic linkages. The number of monosaccharide units can vary; the term polysaccharide is usually reserved for large glycans with more than 20 monosaccharide units. In general an oligosaccharide has less than 20 monosaccharide units.

In a further embodiment of this aspect of the invention the carbohydrate portion of the arrayed reducing end-tagged carbohydrate molecules is a monosaccharide. By ‘monosaccharide’ we mean carbohydrate that cannot be hydrolyzed into a simpler carbohydrate, ie they are the building block of oligosaccharides and polysaccharides. Simple monosaccharides are polyhydroxyaldehydes or polyhydroxyketones with three or more carbon atoms.

In a further embodiment of this aspect of the invention the carbohydrate portion of the arrayed reducing end-tagged carbohydrate molecules is a N-glycan. This carbohydrate is a glycan which on glycoproteins is covalently linked to an asparagine residue of a polypeptide chain in the consensus sequence: -Asn-X-Ser/Thr.

In a further embodiment of this aspect of the invention the carbohydrate portion of the arrayed reducing end-tagged carbohydrate molecules is an O-glycan. This carbohydrate is a glycan which on glycoproteins is covalently linked to the hydroxyl group of the amino acids eg serine and threonine. Where the oligosaccharride is O-glycan, in a further embodiment of this aspect of the invention the O-glycan terminates, for example, in N-acetylgalactosamine, N-acetylgalactosaminitol, mannose or mannitol.

In a further embodiment of this aspect of the invention the carbohydrate portion of the arrayed reducing end-tagged carbohydrate molecules is a fragment of glycosaminoglycan.

The carbohydrate of the arrayed reducing end-tagged carbohydrate molecule of the first aspect of the invention can be prepared from a broad range of sources. The methods used for isolating oligosaccharide or monosaccharides and the subsequent preparation of neoglycolipids is outlined in Fukui et al (2002) Nature Biotech 20, 1011-1017 and WO 87/02777, both incorporated herein by reference.

Hence a further embodiment of the first aspect of the invention is where the arrayed reducing end-tagged carbohydrate molecule has an oligosaccharide or monosaccharide derived from one or more carbohydrate sources selected from glycoproteins, glycolipids, GPI-linked glycans, proteoglycans/glycosaminoglycans and polysaccharides or is synthesised chemically.

In a further embodiment of the first aspect of the invention the arrayed reducing end-tagged carbohydrate molecule is derived from a reducing sugar. In a further embodiment of the first aspect of the invention the arrayed reducing end-tagged carbohydrate molecule is derived from a reduced sugar.

Where the oligosaccharide or monosaccharide is a reduced sugar, a further embodiment of the first aspect of the invention is where the arrayed reducing end-tagged carbohydrate molecule is an oligosaccharide or monosaccharide tagged at the reduced terminal after a mild oxidation procedure as described in WO 87/02777 and Stoll et al (1990) Eur J Biochem 189, 499-507.

As noted above, the arrayed reducing end-tagged carbohydrate molecules of the first aspect of the invention can have one or more samples of immobilised reducing end-tagged carbohydrate molecules in which the carbohydrates within a sample are the same. Alternatively, the reducing end-tagged carbohydrate molecules within a sample may have carbohydrates which are different.

Thus, in a further embodiment of the first aspect of the invention the array has one or more samples of reducing end-tagged carbohydrate molecules which comprise a homogeneous sample of carbohydrate. Therefore, each sample of reducing end-tagged carbohydrate molecules contains a single molecular species of carbohydrate.

Such an embodiment of the invention can be used, for example, to detect whether a specific carbohydrate-interacting molecule is present in a test sample. This will be further detailed below. It can also be used to determine the binding specificity of a specific carbohydrate-interacting molecule.

In a further embodiment of the first aspect of the invention the array has one or more samples of reducing end-tagged carbohydrate molecules which comprise a heterogeneous sample of carbohydrate. Therefore, each sample of reducing end-tagged carbohydrate molecules is a complex mixture of different molecular species of reducing end-tagged carbohydrate molecules.

Such an embodiment of the invention can be used, for example, to detect which general type or specific carbohydrate a carbohydrate-interacting molecule can interact with. This will be further detailed below.

A further embodiment of the first aspect of the invention is where the carbohydrate portion of the arrayed reducing end-tagged carbohydrate molecules is derived from a microbe. For example, the carbohydrate may be obtained from a bacterium, a fungus, or a virus. Such embodiments can be of particular use, for example, in identifying molecules which interact with carbohydrates from microbes. Hence an embodiment of the first aspect of the invention is an array of reducing end-tagged carbohydrate molecules which comprise a carbohydrate derived from a microbe.

A further embodiment of the first aspect of the invention is where the carbohydrate portion of the arrayed reducing end-tagged carbohydrate molecules is derived from a specific cell type. For example, the carbohydrate may be obtained from cancer cells. Such embodiments can be of particular use, for example, in identifying molecules which interact with carbohydrates from cancerous cells. Hence an embodiment of the first aspect of the invention is an array of reducing end-tagged carbohydrate molecules which comprise a carbohydrate derived from a specific cell type.

A further embodiment of the first aspect of the invention is where the carbohydrate portion of the arrayed reducing end-tagged carbohydrate molecules is derived from a specific tissue or organ. For example, the carbohydrate may be obtained from brain, spleen or muscle. Such embodiments can be of particular use, for example, in identifying molecules which interact with carbohydrates from the tissues. Hence an embodiment of the first aspect of the invention is an array of reducing end-tagged carbohydrate molecules which comprise a carbohydrate derived from a specific tissue or organ.

A further embodiment of the first aspect of the invention is where the carbohydrate portion of the arrayed reducing end-tagged carbohydrate molecules is derived from a cell type or tissue from an animal, for example a mouse, rat, rabbit, dog, cat, chimpanzee or human. Preferably, said animal is a human. Such embodiments can be of particular use, for example, in identifying molecules which interact with carbohydrates from a cell type or tissue or organ of a human. Hence an embodiment of the first aspect of the invention is an array of reducing end-tagged carbohydrate molecules which comprise a carbohydrate derived from an animal, for example a human.

A further embodiment of the first aspect of the invention is where the carbohydrate portion of the arrayed reducing end-tagged carbohydrate molecules is derived from a cell type or tissue or organ from a plant. For example, the carbohydrate may be obtained from Arabidopsis thaliana, maize, wheat or rice. Such embodiments can be of particular use, for example, in identifying molecules which interact with carbohydrates from a cell type or tissue of a plant. Hence an embodiment of the first aspect of the invention is an array of reducing end-tagged carbohydrate molecules which comprise a carbohydrate derived from a cell type or tissue from a plant.

In the invention described herein the reducing end-tagged carbohydrate molecules are arrayed on a support. By ‘support’ we mean any material which is suitable to be used to array the reducing end-tagged carbohydrate molecules. Support materials which may be of use in the invention include hydrophobic membranes, for example, nitrocellulose, PVDF or nylon membranes.

Therefore, in further embodiments of this aspect of the invention the support is or comprises a hydrophobic membrane, for example nitrocellulose, PVDF or nylon membranes.

Such membranes are well known in the art and can be obtained from, for example, Bio-Rad, Hemel Hempstead, UK. As will be shown in the accompanying examples, hydrophobic membranes can be successfully used to support the arrayed reducing end-tagged carbohydrate molecules.

A further embodiment of the invention the support on which are arrayed reducing end-tagged carbohydrate molecules comprises a metal oxide.

This embodiment of the invention has arrayed reducing end-tagged carbohydrate molecules presented in a manner that allows other molecules to bind the arrayed reducing end-tagged carbohydrate molecules.

A metal oxide is considered to provide suitable chemical properties for arraying reducing end-tagged carbohydrate molecules. Examples of metal oxides that may be suitable for this aspect of the invention include titanium oxide, tantalum oxide and aluminium oxide. Examples of such materials may be obtained from Sigma-Aldrich Company Ltd, Fancy Road, Poole, Dorset. BH12 4QH UK.

In a further embodiment of the invention such a support is or comprises a metal oxide gel. A metal oxide gel is considered to provide a large surface area within a given macroscopic area to aid immobilisation of the carbohydrate-containing molecules. In a further embodiment of this aspect of the invention such a metal oxide is aluminium oxide.

Additional support materials which may be used on which to array the reducing end-tagged carbohydrate molecules include a gel, for example a silica gel or an aluminium oxide gel. Examples of such materials may be obtained from, for example, Merck KGaA, Darmstadt, Germany.

Included in these embodiments of the invention is an array of reducing end-tagged carbohydrate molecules on a support material (which may be a composite) that can resist change in size or shape during normal use. Examples of support materials are given herein. Composite materials may comprise a component material which can act to provide solidity to the support as well as a component material suitable to array the reducing-end tagged carbohydrate molecules. For example the support may be a glass slide coated with a component material suitable to be used to array the reducing end-tagged carbohydrate molecules.

The molecules may be immobilised via non-covalent interactions: hydrogen bonding and ionic bonding and van der Waals interaction.

As discussed above, the first aspect of the invention comprises an array of reducing end-tagged carbohydrate molecules immobilised on a support. It is generally necessary to first solubilise the reducing end-tagged carbohydrate molecules to allow them to be immobilised on the support. However, some reducing end-tagged carbohydrate molecules are poorly soluble in water, for example, glycolipids and neoglycolipids. Hence it may be necessary or desirable to use other solvents to dissolve or suspend these reducing end-tagged carbohydrate molecules so they can be immobilised on the support.

Currently, the solvents used to solubilise neoglycolipids and glycolipids are organic-based solvent mixtures usually containing chloroform (see, for example, Fukui et al (2002) Nat Biotech 20, 1011-1017). Chloroform is often an essential component of the solvent but it can be problematic as it is highly volatile. For example, the chloroform/methanol/water 25:25:8 by volume solvent used by Fukui et al (2002) Nat Biotech 20, 1011-1017 cannot be used in some modem arrayers of either contact or non-contact types.

We have developed alternative aqueous/low volatility solvents that can be used to solubilise neoglycolipids and glycolipids. Such a solvent may be of use in, for example, preparing an array of reducing end-tagged carbohydrate molecules according to the first aspect of the invention. The solvents are comprised of aqueous/aliphatic alcohol mixtures.

Hence a further aspect of the invention is a method of preparing an array according to the first aspect of the invention wherein the reducing end-tagged carbohydrate molecule is immobilised on the support while solubilised in a solvent comprising an aqueous/aliphatic alcohol mixture. Preferably, the reducing end-tagged carbohydrate molecule is a neoglycolipid or a glycolipid.

A further embodiment of this aspect of the invention is where the solvent includes an aliphatic alcohol selected from a list comprising: propanol (propan-1-ol), iso-propanol (propan-2-ol), n-butanol (butan-1-ol), iso-butanol (butan-2-ol), and t-butanol (2-methylpropan-2-ol). Preferably, the aliphatic alcohol constitutes between 4, 5, 6, 7, 8, 9, 10, 11, 12, 13, 14, 15, 16, 17, 18, 19, 20, 25 or 30% of the solvent by volume. More preferably, the aliphatic alcohol constitutes between 8 to 15% (v/v) of the solvent.

While not wishing to be bound to a particular theory, it is thought that the reducing end-tagged carbohydrate molecule or molecules are immobilised on a support according to the first aspect of the invention via a non-covalent interaction, such as hydrogen bonding, ionic or van der Waals interaction.

A further aspect of the invention is a method for detecting a molecule in a test sample comprising:

i) contacting an array according to the first aspect of the invention with the test sample; and,

ii) detecting the binding of any molecules in the test sample to the array.

By ‘an array’ we include all embodiments of the array as set out in the first aspect of the invention, including where the reducing end-tagged carbohydrate molecules are neoglycolipids

By ‘a test sample’ we include a sample of a body fluid such as blood, serum, plasma, urine, cerebrospinal fluid, pleural fluid and semen.

The binding of a molecule in the test sample to the arrayed reducing end-tagged carbohydrate molecules can be measured using, for example, colorimetric or fluorescence detection systems, or other labelling methods, or other methods that do not require labelling.

Such a method may be of particular use in, for example, detecting the presence of a specific protein in serum, eg antibody, by its binding to an arrayed reducing end-tagged carbohydrate molecules. In this way it could be determined whether a patient has contracted a disease caused by a certain microbe or parasite. An example of how such a method could be used is disclosed in Example 5.

A further aspect of the invention is a method of determining whether a molecule interacts with a carbohydrate comprising:

i) contacting an array according to the first aspect of the invention with the molecule; and

ii) measuring whether the molecule binds to any of the arrayed reducing end-tagged carbohydrate molecules.

By ‘an array’ we include all embodiments of the array as set out in the first aspect of the invention, including where the reducing end-tagged carbohydrate molecules are neoglycolipids.

The binding of a molecule in the test sample to the arrayed reducing end-tagged carbohydrate molecules can be measured using, for example, calorimetric or fluorescence detection systems, or other labelling methods, or other methods that do not require labelling.

Such a method may be of particular use in, for example, identifying whether a molecule thought to be capable of interacting with a carbohydrate can actually do so, or to identify whether a molecule unexpectedly has the capability of interacting with a carbohydrate.

Also included in this method is where the array is an array of heterogeneous reducing end-tagged carbohydrate molecules. In this method a molecule can be assayed to determine whether it interacts with carbohydrates present in, for example, a human cell or tissue type. Such a method may be of use in screening possible therapeutic molecules to identify those molecules which bind to reducing end-tagged carbohydrate molecules. An example of how such a method could be used is disclosed in Example 6.

A further aspect of the invention is a method of determining the kinetics of interaction between a molecule and a carbohydrate comprising:

i) contacting an array according to the first aspect of the invention with a molecule; and,

ii) measuring the kinetics of interaction between the molecule and the reducing end-tagged carbohydrate molecules on the support.

We include all embodiments of the array as set out in the first aspect of the invention.

The kinetics of interaction of a molecule to any of the arrayed reducing end-tagged carbohydrate can be measured by real time changes in, for example, colorimetric or fluorescent signals.

Such a method may be of particular use in, for example, determining whether a molecule is able to interact with a specific carbohydrate with a higher degree of binding than a different molecule to the same carbohydrate. This may be of use in identifying a therapeutic molecule which can bind with a high affinity to an arrayed reducing end-tagged carbohydrate molecule. An example of how such a method could be used is disclosed in Example 7.

A further aspect of the invention is a method of identifying a carbohydrate-binding molecule or molecules in a heterogeneous sample of molecules comprising:

i) contacting an array according to the first aspect of the invention with a heterogeneous sample of molecules; and

ii) identifying those molecules which interact with any of the arrayed reducing end-tagged carbohydrate molecules.

By ‘an array’ we include all embodiments of the array as set out in the first aspect of the invention, including where the reducing end-tagged carbohydrate molecules are neoglycolipids.

The binding of a molecule in the test sample to the arrayed reducing end-tagged carbohydrate molecules can be measured using, for example, calorimetric or fluorescence detection systems, or other labelling methods, or other methods that do not require labelling.

Such a method may be of particular use in, for example, identifying whether any molecules present in a test sample are capable of interacting with any of the arrayed reducing end-tagged carbohydrate molecules. Also included in this method is where the array is an array of heterogeneous carbohydrate molecules. In this method a heterogeneous population of molecules can be assayed to identify those molecules that interact with carbohydrates present in, for example, a human cell or tissue type. Such a method may be of use in identifying possible therapeutic molecules. An example of how such methods can be used is disclosed in Example 8.

A further aspect of the invention is a method of identifying a carbohydrate bound by a molecule (optionally from a heterogeneous population of molecules) comprising:

i) contacting an array according to the first aspect of the invention, with a quantity of the molecule; and

ii) identifying the reducing end-tagged carbohydrate molecule or molecules on the array to which the molecule binds.

By ‘an array’ we include all embodiments of the array as set out in the first aspect of the invention, including where the reducing end-tagged carbohydrate molecules are neoglycolipids.

The binding of a molecule in the test sample to the arrayed reducing end-tagged carbohydrate molecules can be measured using, for example, calorimetric or fluorescence detection systems, or other labelling methods, or other methods that do not require labelling.

Such a method may be of particular use in, for example, identifying which reducing end-tagged carbohydrate molecule or molecules (optionally in a heterogeneous population of such molecules) are able to bind to a test sample or a molecule. For example, if it is known that a specific molecule interacts with a carbohydrate-containing molecule in a cell then the method can be used to identify the specific carbohydrate-containing molecule to which the molecule binds. An example of how such a method could be used is disclosed in Example 9.

In a further embodiment of this aspect of the invention the method further comprises a deconvolution process. The purpose of a deconvolution process is to identify specific reducing end-tagged carbohydrate molecules, to which a molecule binds, in a complex mixture of reducing end-tagged carbohydrate molecules. One such method involves: isolating a complex mixture of reducing end-tagged carbohydrate molecules to which a test sample or molecule binds, making a ‘daughter’ array of the complex mixture of reducing end-tagged carbohydrate molecules separated into individual or a restricted number of molecules, binding the test sample or molecule to the ‘daughter’ array, and identifying the reducing end-tagged carbohydrate molecules to which the test sample or molecule binds, using mass spectrometry preceded as necessary by thin-layer or multi-dimentional chromatographies and chromatogram binding.

A further aspect of the invention is a method of separating specific cells from a heterogeneous population of cells comprising:

i) providing an array according to the first aspect of the invention, wherein at least some of the reducing end-tagged carbohydrate molecules are able to interact with the specific cells;

ii) contacting the array with a heterogeneous population of cells; and

iii) separating those cells that bind to the array from those cells that do not bind to the array.

By ‘an array’ we include all embodiments of the array as set out in the first aspect of the invention, including where the reducing end-tagged carbohydrate molecules are neoglycolipids.

Such a method may be of particular use in, for example, identifying cells that have on their surface a molecule which binds to arrayed reducing end-tagged carbohydrate molecules. For example, the method may be of use in panning experiments to isolate cells having specific molecules on their surface, as will be appreciated by those skilled in the art. The method may also be of use in identifying microbes from the urine of a patient having, or suspected of having, a urinary infection caused by a bacterium. An example of how such methods can be used is disclosed in Example 10.

A further aspect of the invention is a method of determining whether a test molecule interferes with the binding of a molecule or cell to a reducing end-tagged carbohydrate molecule comprising:

i) preparing an array according to the first aspect of the invention, wherein at least some of the reducing end-tagged carbohydrate molecules are bound by a molecule or cell;

ii) contacting the array with a quantity of the molecule; and

iii) identifying whether the molecule interferes with the binding of a molecule or cell to an arrayed reducing end-tagged carbohydrate molecule.

By ‘an array’ we include all embodiments of the array as set out in the first aspect of the invention, including where the reducing end-tagged carbohydrate molecules are neoglycolipids.

The binding of a molecule in the test sample to the arrayed reducing end-tagged carbohydrate molecules can be measured using, for example, calorimetric or fluorescence detection systems, or other labelling methods, or other methods that do not require labelling.

Such a method may be of particular use to, for example, identify whether a molecule, for example a small drug or carbohydrate-mimic, can interfere with the binding of a molecule to a carbohydrate. This could be the basis of a competition/inhibition assay screen as would be appreciated by a person skilled in the art. An example of how such a method could be used is disclosed in Example 11.

In a further embodiment of this aspect of the invention the test molecule is part of a heterogeneous population of molecules.

Such a method may be of particular use, for example, to identify which molecules in a heterogeneous population of molecules can interfere with the binding of another molecule to a carbohydrate. An example of how such a method could be used is disclosed in Example 11.

In a further embodiment of the methods of the invention, the molecule that may interact with a reducing end-tagged carbohydrate molecule, or a test molecule that may interfere with the binding of a molecule to a reducing end-tagged carbohydrate molecule, may be a polypeptide, for example, an antibody, enzyme, receptor, lectin or glycoprotein. The molecule may also be a peptidomimetic, nucleic acid, carbohydrate, lipid, glycolipid, hormone, microbial antigen or glycomimic. The molecule may also be a therapeutic molecule, for example, a therapeutic molecule smaller than 500 daltons, a prophylactic, a vaccine, or an immunomodulator.

The term “peptidomimetic” refers to a compound that mimics the conformation and desirable features of a particular peptide as a therapeutic agent, but that avoids the undesirable features. For example, morphine is a compound which can be orally administered, and which is a peptidomimetic of the peptide endorphin. There are a number of different approaches to the design and synthesis of peptidomimetics, such as those discussed in Sherman and Spatola, J. Am. Chem. Soc., 112: 433 (1990), Meziere et al (1997) J. Immunol. 159 3230-3237, Veber et al, Proc. Natl. Acad. Sci. USA, 75:2636 (1978), Thursell et al, Biochem. Biophys. Res. Comm., 111:166 (1983) and D. H. Rich in Protease Inhibitors, Barrett and Selveson, eds., Elsevier (1986), all incorporated herein by reference.

A further aspect of the invention is a molecule as identified by any of the methods of the invention disclosed above.

A further aspect of the invention is a carbohydrate as identified by any of the methods of the invention disclosed above.

As discussed above we have developed alternative aqueous/low volatility solvents that can be used to solubilise neoglycolipids and glycolipids. The solvents are comprised of aqueous/aliphatic alcohol mixtures. Such solvents can be used solubilise neoglycolipids and glycolipids prior to preparing an array according to the first aspect of the invention.

Hence a further aspect of the invention is the use of a solvent comprising an aliphatic alcohol for solubilising a reducing end-tagged carbohydrate molecule in the preparation of an array according to the first aspect of the invention. Preferably, the reducing end-tagged carbohydrate molecule is a neoglycolipid or a glycolipid.

In this aspect of the invention a reducing end-tagged carbohydrate molecule is first solubilised using a solvent comprising an aliphatic alcohol. The solubilised reducing end-tagged carbohydrate molecule is then arrayed on the support.

An advantage of using such a solvent over existing solvents for solubilising a reducing end-tagged carbohydrate molecule is that a solvent comprising an aliphatic alcohol is less volatile and can be used in some modern arrayers of either contact or non-contact types.

A further embodiment of this aspect of the invention is where the solvent used has an aliphatic alcohol selected from a list comprising: propanol (propan-1-ol), iso-propanol (propan-2-ol), n-butanol (butan-1-ol), iso-butanol (butan-2-ol), and t-butanol (2-methylpropan-2-ol). Preferably, the aliphatic alcohol constitutes between 4, 5, 6, 7, 8, 9, 10, 11, 12, 13, 14, 15, 16, 17, 18, 19, 20, 25 or 30% of the solvent by volume. More preferably, the solvent constitutes between 8 to 15% (v/v) of the solvent.

Any publications referred to herein are hereby incorporated by reference.

The invention will now be described in more detail by reference to the following non-limiting Figures and Examples.

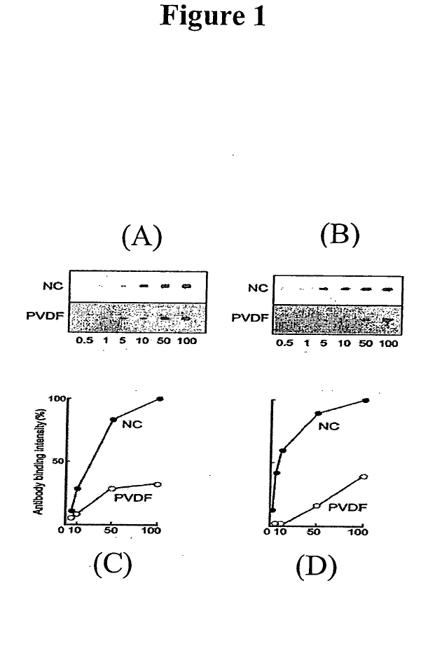

FIG. 1: Immunological detection of oligosaccharides immobilized as NGLs on nitrocellulose and PVDF membranes.

(A and B) The fluorescent NGLs of LNFP III and CSC 16mer were printed onto nitrocellulose (NC) and PVDF membranes at the levels indicated, and immunostained with anti-SSEA-1, and anti-CS(CS-56), respectively. (C and D) Quantitative illustration of the experiments in panels A and B, showing the relative intensities of the binding signals with anti-SSEA-1 and CS-56, respectively, when the NGLs of LNFP III and CSC 16mer were printed onto nitrocellulose and PVDF membranes. The intensity of antibody binding was measured by scanning with a Shimadzu CS 9000 densitometer. The intensity of binding when 100 pmol of the NGL was applied onto nitrocellulose was taken as 100%.

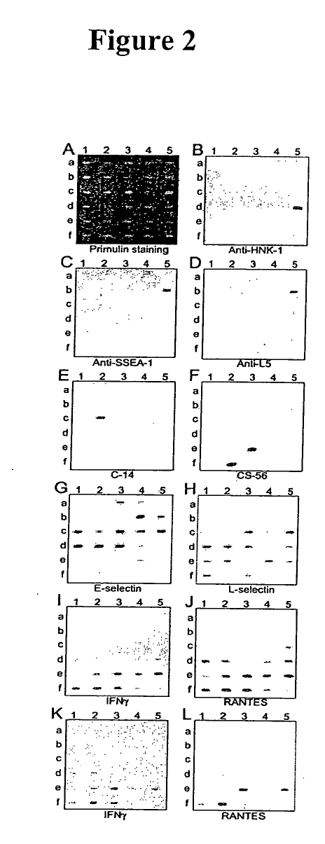

FIG. 2: Detection of epitope or ligand-bearing oligosaccharides on nitrocellulose membranes probed with carbohydrate-recognizing proteins.

Thirty lipid-linked oligosaccharides (IDs 1-30, Table 1) were printed onto a silica gel plate (A) for detection by primulin staining, and onto nitrocellulose membranes (B-L) for probing with the monoclonal antibodies (HNK-1, anti-SSEA-1, anti-L5, C-14 and CS-56), the E- and L-selectins, the cytokine INFY and the chemokine RANTES. Printing was at 100 pmol per spot in panel A, 10 pmol (B-J) and 1 pmol (K, L).

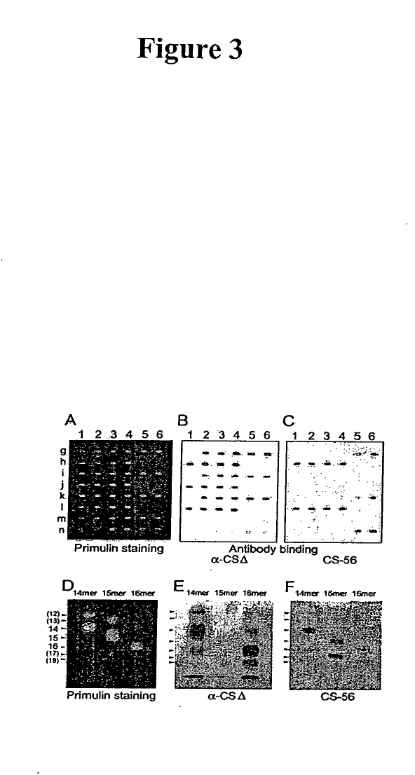

FIG. 3: Surveying the features of antigen-positive oligosaccharides within arrays derived from chondroitin sulfates A, B and C.

NGLs of forty-two oligosaccharide fractions (IDs 22-27, 31-66, prepared from chondroitin sulfates A, B and C were printed at 100 pmol onto a silica gel plate and primulin stained (panel A), and at 10 pmol onto nitrocellulose membranes and immunostained with α-CSΔ (panel B) and CS-56 (panel C). The predominant sequences and locations of the oligosaccharides are given in Table 1.

In panel D, the primulin staining and in E and F, immunostainings are shown of chromatograms of the 14mer and 16mer fractions, IDs 27 and 52, of chondroitin sulfate C, and of the 15mer fraction obtained after removal of the terminal unsaturated uronic acid from the 16mer fraction. TLC was performed on aluminum-backed silica gel plates using a solvent mixture CHCl₃/MeOH/0.2% CaCl₂, 50:60:20 (v/v). Arrowheads indicate the positions of migration of the 14, 15, and 16mers, and in parentheses, those of minor components 12, 13, 17 and 18mers.

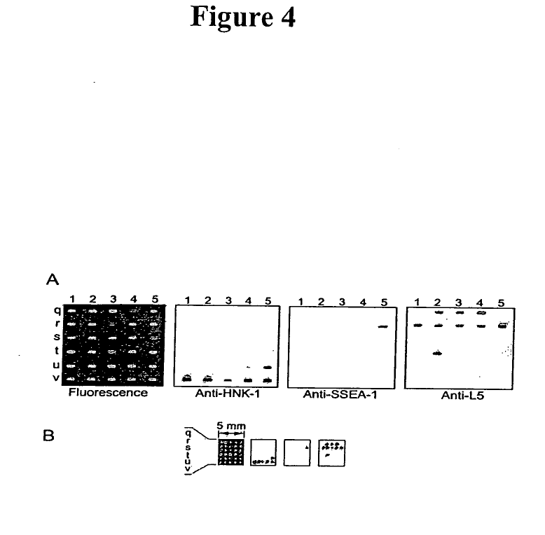

FIG. 4: Detection of the selective expression of carbohydrate differentiation antigens in brain O-glycan arrays.

Fluorescent NGLs of brain-derived O-glycan alditols, tri- to octasaccharides, separated into ten neutral, fourteen sialyl and six sulfated fractions (Table 1) were printed onto PVDF membranes for fluorescenc detection, and onto nitrocellulose for probing with monoclonal antibodies: anti-HNK-1, anti-SSEA-1 and anti-L5. In A, the NGLs were printed at 10 pmol and in B, at 2 pmol per spot.

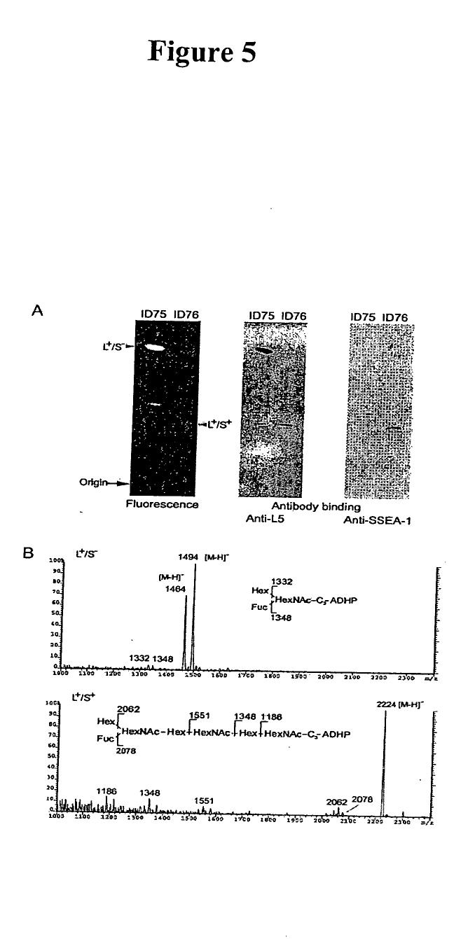

FIG. 5: Deconvolution by TLC, antibody binding and mass spectrometry to examine the features of two distinct Lewis^{x}-bearing oligosaccharides.

(A) Fluorescence detection, after resolution by TLC of the fluorescent NGLs in fractions IDs 75 (r4) and 76 (r5), and immunodetection of the L5-positive/SSEA-1-negative components (main component designated L⁺/S⁻) and the doubly positive component (L⁺/S⁺). The TLC of NGLs was performed on aluminum-backed silica gel plates using a solvent mixture CHCl₃/MeOH/H₂O, 60:35:8 (v/v). (B) Sequence determination of components L⁺/S⁻ and L⁺/S⁺by in situ liquid secondary-ion mass spectrometric analyses in the negative-ion mode. Two components were identified in L⁺/S⁻ and a single component was identified in L⁺/S⁺. In the spectrum of L⁺/S⁻, the sequence and fragment ions are shown for the major component containing a three-carbon core fragment, [M−H]⁻ at m/z 1494. The minor component, [M−H]⁻ m/z 1464 contained a two carbon fragmment. Hex: hexose; HexNAc, N-acetylhexosamine.

FIG. 6: Schematic of arrangements in which reducing end-tagged carbohydrate molecules may be arrayed.

Diagram shows a support according to the first or second aspects of the invention (represented by a rectangle) on which are a number of locations at which reducing end-tagged carbohydrate molecules may be immobilised (represented by black spots). Each location can be specified according to its position on the support, e.g. A1, A2, B1, B2 etc.

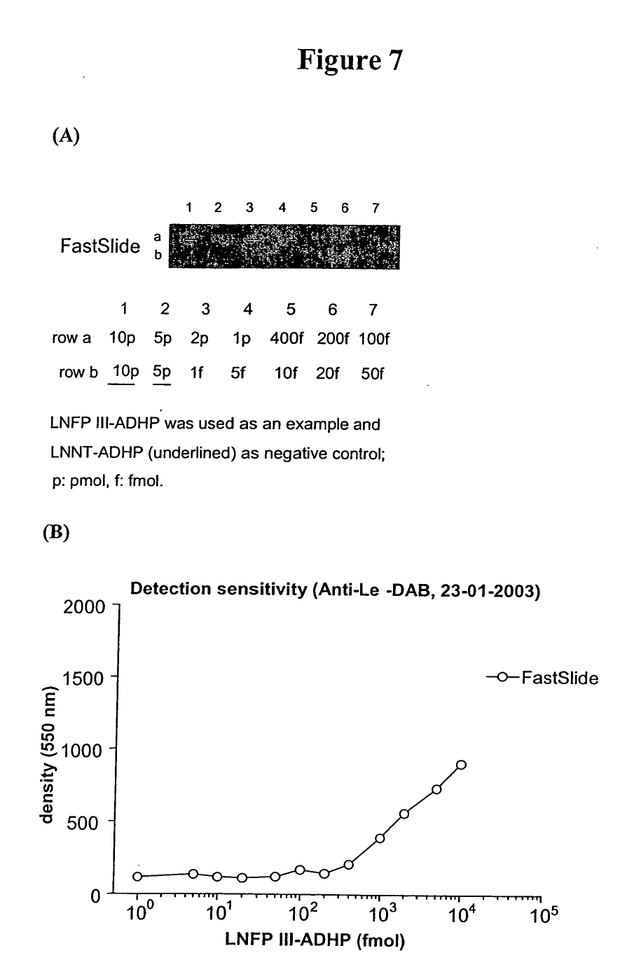

FIG. 7: Detection sensitivity of NGLs on a nitrocellulose-coated FastSlide by anti-Le^{x} antibody.

In (A) different amounts (1 fmol to 10 pmol) of fluorescent NGLs were applied as 2 mm bands on a nitrocellulose coated FastSlide. The Le^{x} pentasaccharide LNFP III-ADHP was used as an example to demonstrate the detection sensitivity by an anti-Le^{x} antibody; a tetrasaccharide LNNT-ADHP (positions 1b and 2b) was used as the negative control. Binding was revealed by the development of DAB colour reaction. In (B) the binding intensity revealed by the DAB colour of each bands was scanned at 550 nm, recorded and plotted for direct comparison.

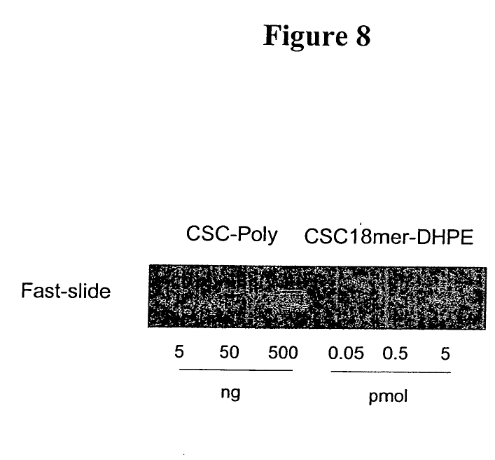

FIG. 8: Anti-chondroitin sulphate (CS) antibody binding to CSC polysaccharide and CSC 18mer-DHPE neoglycolipid.

Different amounts of CSC polysaccharide (5 to 500 ng) and its oligosaccharide derived NGL, CSC 18mer-DHPE (0.05 to 5 pmol), were applied as 2 mm bands on a nitrocellulose coated FastSlide. Binding was revealed by the development of DAB colour reaction.

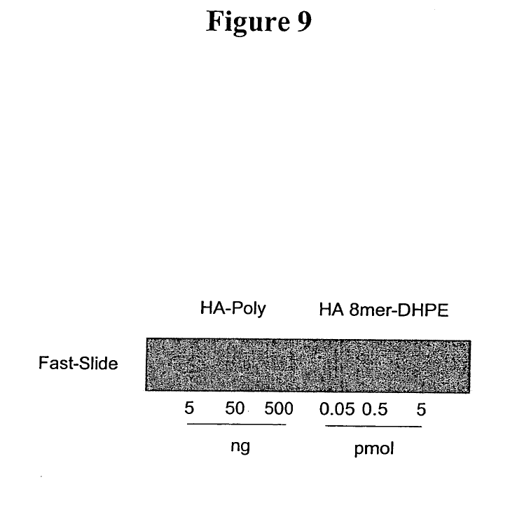

FIG. 9: Protein TSG-6 binding to hyaluronic acid (HA) and HA 8mer-DHPE neoglycolipid.

Different amounts of HA polysaccharide (5 to 500 ng) and its oligosaccharide derived NGL, HA 8mer-DHPE (0.05 to 5 pmol), were applied as 2 mm bands on a nitrocellulose coated FastSlide. Binding was revealed by the development of DAB colour reaction.

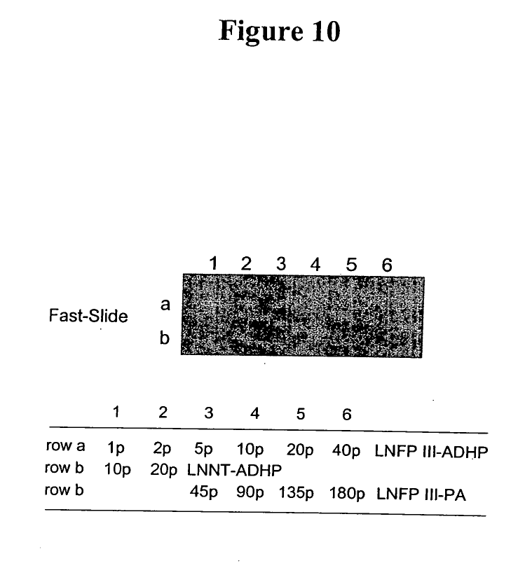

FIG. 10: Binding of anti-Le^{x} antibody to LNFP III as aminopyridine and ADHP derivatives a nitrocellulose-coated FastSlide.

Different amounts of oligosaccharide derivatives were applied as 2 mm bands on a nitrocellulose coated FastSlide. The ADHP (bands 1a-6a) and aminopyridine (PA) derivative (bands 3b-6b) of the Le^{x} pentasaccharide LNFP III were detected by an anti-Le^{x} antibody. The tetrasaccharide LNNT-ADHP was used as negative control (positions 1b and 2b). Binding was revealed by the development of DAB colour reaction.

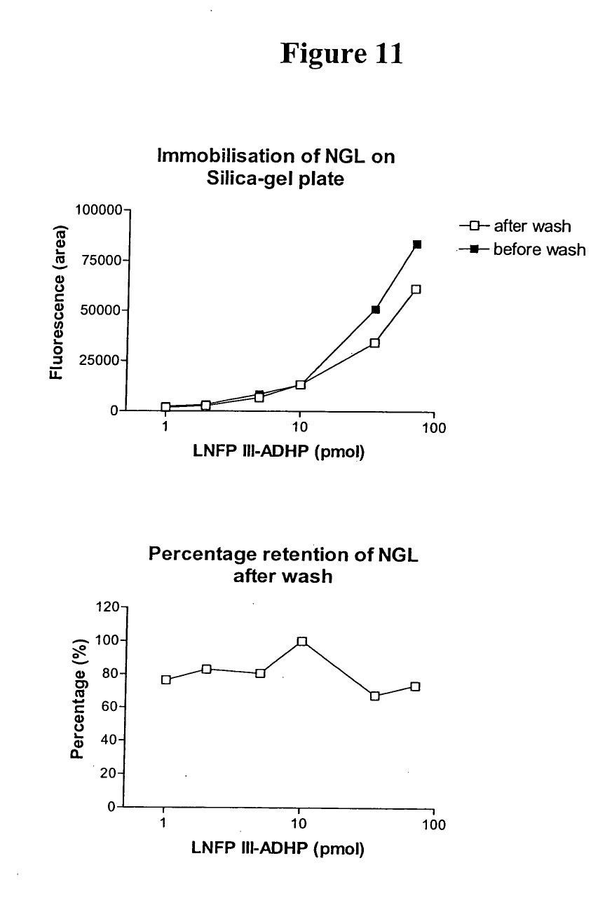

FIG. 11: Immobilisation of NGLs on a silica gel glass slide.

Different amounts of the fluorescent neoglycolipid LNFP III-ADHP were applied on a silica gel-coated glass slide in 2 mm bands (1, 2, 5, 10, 35 and 70 pmol). After washing with PBS at room temperature for 3 hours, the retained NGL on the matrix was measured by its fluorescence and compared with freshly applied unwashed bands. The fluorescence intensities were scanned and shown in the upper panel, and the percentage of NGL retained shown in the lower panel.

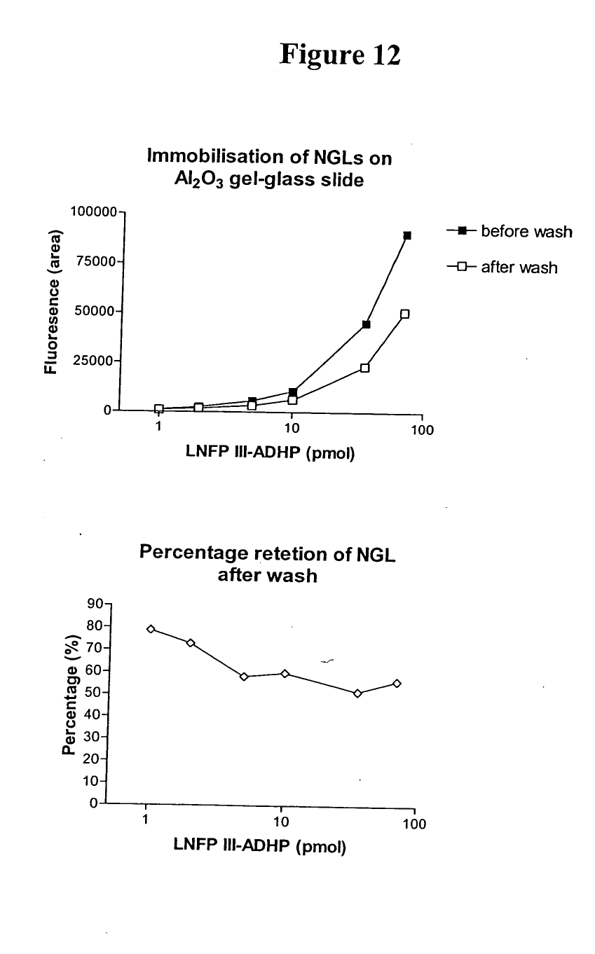

FIG. 12: Immobilisation of NGLs on aluminium oxide gel glass slide.

Different amounts of the fluorescent neoglycolipid LNFP III-ADHP were applied on an aluminium oxide gel glass slide as 2 mm bands (1, 2, 5, 10, 35 and 70 pmol). After washing with PBS at room temperature for 3 hours, the retained NGL on the matrix was measured by its fluorescence and compared with freshly applied unwashed bands. The fluorescence intensities were scanned and shown in the upper panel, and the percentage of NGL retained shown in the lower panel.

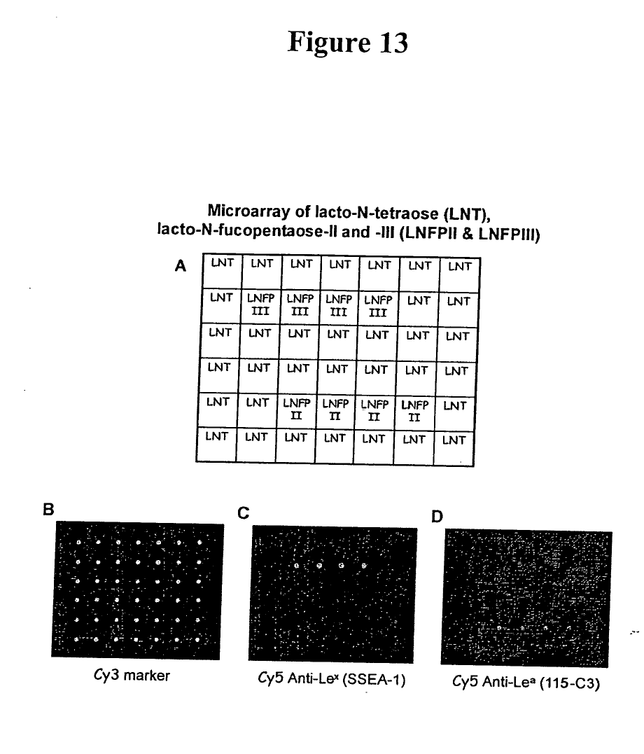

FIG. 13: Microarray of NGLs on a nitrocellulose-coated FastSlide

NGLs of LNFP III-DHPE, LNFP II-DHPE and LNT (50 finol per spot), in 20% n-propanol in water, were arrayed (layout is in panel A) with a touch-type pin microarrayer in the presence of Cy3 dye as a marker (panel B). One slide was stained with an ant-Le^{x} antibody (panel C) and another with an anti-Le^{a} (panel D). Binding was detected with a biotinylated anti-mouse followed byCy5-labelled streptavidin. The LNT was included as a negative control.

### EXAMPLE 1

### Oligosaccharide Microarrays Toward High-Throughput Detection and Specificity Assignments of Carbohydrate-Protein Interactions

Abstract

We describe microarrays of oligosaccharides as neoglycolipids and their robust display on nitrocellulose. The unique feature is that arrays are sourced from glycoproteins, glycolipids, proteoglycans, polysaccharides, whole organs or from chemical syntheses. We show that carbohydrate-recognizing proteins single-out their ligands, not only in the arrays of homogenous oligosaccharides, but also in arrays of heterogeneous oligosaccharides. Deconvolution strategies are included with mass spectrometry for sequencing ligand-positive components within mixtures. New findings in initial applications include: (a) among O-glycans in brain, a relative abundance of the Lewisx sequence based on N-acetyllactosamine, recognized by anti-L5, and a paucity based on poly-N-acetyllactosamine, recognized by anti-SSEA-1, (b) insights into chondroitin sulfate oligosaccharides recognized by an antiserum and an antibody (CS-56) to chondroitin sulfates, and (c) binding of the cytokine interferon γ and the chemokine RANTES to sulfated sequences such as HNK-1, sulfo-Lewis^{x} and sulfo-Lewis^{a}, in addition to glycosaminoglycans. This approach opens the way for discovering new carbohydrate-recognizing proteins in the proteome, and mapping the repertoire of recognition structures in the glycome.

Introduction

Knowledge that the human genome encodes no more than 30-50 thousand proteins has served to emphasize the importance of post-translational modifications as modulators of the activities and functions of proteins in health and disease. One such modification, par excellence, is glycosylation. Moreover, information is increasing on proteins that act through oligosaccharide-recognition; these mediate critical processes such as protein folding and trafficking, and play key roles in mechanisms of immunity and microbe-host interactions²⁻⁵. However, the pinpointing and elucidation of the recognition elements on oligosaccharide chains remain among the most challenging areas of cell biology. This is because oligosaccharides of glycoproteins are diverse and typically very heterogeneous. They cannot be readily cloned, being the products of numerous glycosyltransferases. There is a great need, therefore, to design microarray technologies for oligosaccharides that would perrnit systematic and high-throughput analyses of protein-carbohydrate interactions⁶, analogous to those developed for DNA⁷, and being developed for proteins⁸. Approaches have been established recently for microarrays of polysaccharides⁹, and monosaccharides¹⁰ but not for the oligosaccharide sequences found on polysaccharides, and on glycoproteins and proteoglycans. This is an important requirement for delineating the recognition elements on glycoconjugates. The neoglycolipid (NGL) technology for generating lipid-linked oligosaccharide probes from glycoproteins and polysaccharides¹¹⁻¹⁶ is well suited for this challenge. A key feature is that bioactive carbohydrate chains can be singled out from heterogeneous mixtures and characterized by mass spectrometry. NGL technology has been powerful for example in the discovery, on epithelial glycoproteins, of hitherto unsuspected sulfated carbohydrate ligands for the selectins^{17,18}, novel O-mannosyl modifications of brain glycoproteins¹⁹⁻²¹, and a unique antigenic sequence on heparan sulfate²².

We now explore the potential of NGL technology as the basis of a microarray system applicable to oligosaccharides derived not only from biological sources, but also by chemical syntheses, conventional and combinatorial. The technology has the potential for generating large repertoires of irnmobilized oligosaccharide probes required for the discoveries of carbohydrate-protein interactions and assignments of their specificities. We show that NGLs, derived from the oligosaccharides of glycoproteins, glycosaminoglycans or an organ such as the brain, are robust probes when presented on nitrocellulose membranes. They permit sensitive and potentially high throughput detection of ligands for carbohydrate-binding proteins, as exemplified here for antibodies, animal lectins, a chemokine and a cytokine.

Results and Discussion

Detection of Carbohydrate-Binding Signals with NGLs Immobilized on Nitrocellulose and PVDF Membranes.

In initial quantitative immunostaining experiments using anti-SSEA-1 and CS-56, and the NGLs of the neutral oligosaccharide lacto-N-fucopentaose III (LNFP III) and the acidic 16mer from chondroitin sulfate C(CSC), we observed that the intensities of the binding signals were greater on the nitrocellulose than on the PVDF membrane (substantially so in the case of CSC 16 mer), even taking into account the amounts of the NGLs retained on the two membranes (results not shown). For the carbohydrate-binding experiments, therefore, the lipid-linked oligosaccharides were printed onto nitrocellulose. For the fabrication of arrays, unless otherwise stated, 10 pmol was applied per spot, which is on a near linear part of the binding curve.

Preservation of the Binding Specificities of Monoclonal Antibodies and the Selectins Toward Oligosaccharides Arrayed on Nitrocellulose Membranes.

We first investigated whether lipid-linked oligosaccharides arrayed on nitrocellulose are effective probes in the design of arrays to detect protein-carbohydrate interactions. We selected as models seven proteins with known carbohydrate-binding specificities that have been investigated previously by various conventional procedures. They include the monoclonal antibodies anti-HNK-1, anti-SSEA-1, anti-L5, C-14 and CS-56, and the leukocyte-endothelium adhesion molecules, E- and L-selectins. An array of 30 oligosaccharide probes was printed with various sequence features (Table 1, IDs 1-30, and FIG. 2A) including those that are known to be recognized by each of the seven proteins. These carbohydrate-binding proteins indeed singled out their specific epitopes or ligands within the array (FIG. 2B-H), respectively. Four of the monoclonal antibodies each bound to one of the oligosaccharide probes (FIG. 2B-E) in complete accord with previous assignments of specificities: toward the 3-sulfoglucuronyl-lacto-N-neo-lactosamine sequence²³ as on ID 20 for anti-HNK-1, the Lewis^{x} sequence as on ID 10 for anti-SSEA-1^{24,25} and anti-L5²⁶, and the Lewis^{y} sequence, as on ID 12 for C-14 antibody²⁷. The fifth antibody, CS-56, known to recognize chondroitin sulfates A and C²⁸ bound, as predicted, to the NGLs of the CSA and CSC 14mers, IDs 23 and 27, respectively (FIG. 2F). The binding profiles of the selectins were also in accord with earlier assignments^{29,30}; namely, there was an overlap in the E- and L-selectin binding to 3′-sialyl and 3′-sulfated Lewis^{a} and Lewis^{x} sequences, IDs 13-18 (weak L-selectin binding to ID 14), and non-overlapping binding by E-selectin to the neutral fuco-oligosacchairde, ID 3, and to the Lewis^{a}, Lewis^{x}, Lewis^{b} and Lewis^{y} sequences, as in IDs 9-12, and by L-selectin to the pentamannose phosphate, ID 21, and to the non-fucosylated sulfated sequences, IDs 20, 22, 24, 25, 26 and 28.

Insights into Binding Specificities of the Cytokine Interferon γ (IFN-γ and RANTES, and of Antibodies Directed to Chondroitin Sulfates.

When IFN-γ and RANTES were overlaid onto the array, IDs 1-30, binding was observed to several of the sulfated probes (FIG. 2I-L). The repertoire of oligosaccharides bound was narrower when the array was printed at 1 pmol per spot (FIG. 2K, L), with a clear preference for the chondroitin sulfate glycosaminoglycan probes, IDs 23, 25 and 27; and in the case of IFN-γ also for the heparin/heparan sulfate probe, ID 28. This is in accord with reports on their binding to the glycosaminoglycan moieties of proteoglycans^{31,32}.

These are electrostatic interactions believed to be a mechanism for increasing local concentrations of the humoral mediators in tissues, protecting them from degradation and facilitating their binding to their high affinity receptors. At the higher printing level, 10 pmol per spot (FIGS. 2I and J), both proteins gave additional or increased binding signals (more pronounced with RANTES) with several other sulfated probes including CSA 2mer (ID 22) the HNK-1 sequence (ID 20) and the sulfated Lewis^{a/x} sequences (IDs 15-17, 19). The weak binding to 3′-sulfated Lewis^{x} (ID16, d1) and 3′6-sulfated Lewis^{x} (ID 17, d2) was more apparent at 1 pmol loading than at 10 pmol (cf FIGS. 2K and I). Further investigation is required to determine whether this is due to a “prozone” phenomenon (diminished binding) sometimes observed with ligand excess.

We prepared a novel array of 42 chondroitin sulfate probes derived from 2 to 20mer fractions (FIG. 3A and Table 1), and examined their recognition by the polyclonal antiserum, α-CSΔ, and monoclonal CS-56. With A-CSA, the smallest probes bound were those derived from the 4mer fractions of CSA, CSB and CSC, IDs 31, 39 and 47, respectively (FIG. 3B), containing unsaturated uronic acid at their non-reducing ends. The antiserum gave negligible or no binding signals with NGLs derived from the oligosaccharides, IDs 55-60, from which the unsaturated uronic acid had been removed, and none with IDs 61-66, which have an unmodified glucuronic acid at their non-reducing ends. Taking into account the ring-opened state of the monosaccharides joined to the lipid tag¹², these results establish that the antiserum recognizes an unsaturated uronic acid, formed in the course of chondroitin sulfate cleavage with chondroitinase ABC, and at least a subterminal GaINAc residue with an intact pyranose ring.

The determinant on CSA and CSC for monoclonal antibody CS-56 was examined with the array of CS-derived NGLs (FIG. 3C). Binding was detected to those derived from 10mer and higher oligosaccharide fractions, IDs 34-38, and 50-54, in addition to IDs 23 and 25, already shown in FIG. 2. Binding was also detected to the NGLs derived from CSC 12mer and 14mer fractions, IDs 65 and 66, with unmodified terminal glucuronic acid. In separate experiments (FIG. 3D-F), CS-56 bound to the NGLs derived from a CSC 15mer fraction obtained by removal of the terminal unsaturated uronic acid from the 16mer fraction, ID 52. This contrasted with α-CSΔ which gave no binding signal with NGLs in the 15mer fraction. Collectively, these results indicate that the determinant recognized by CS-56 is expressed within a 10mer sequence common to CSA and CSC, and that the antibody combining site is of groove type, recognizing internal sequences in the CS chains.

Probing a Complex O-glycan Array from Brain.

Carbohydrate microarrays from whole cells or organs could be a powerful means of discovering novel ligands for carbohydrate-binding proteins. We generated a brain O-glycan array, derived from tri- to octasaccharide alditols in thirty fractions: ten neutral, fourteen sialylated and six sulfated (Table 1, IDs 67-95). We used this as a model system for high-throughput, high sensitivity detection of carbohydrate binding, followed by deconvolution of the binding elements. Fluorescent NGLs were used to aid the sensitive imaging of the primary array printed onto PVDF membranes and the secondary arrays after resolution by TLC (see below). Two formats were evaluated, a ‘miniarray’ format (FIG. 4A), 2 mm bands of 10 pmol, and a ‘microarray’ format, 300 μm spots of 2 pmol (FIG. 4B).

Three of the monoclonal antibodies to carbohydrate differentiation antigens, HNK-1, anti-SSEA-1 and anti-L5, were used as model carbohydrate-binding proteins to probe the brain O-glycan array; the results were comparable for the two array formats. Binding by anti-HNK-1 was readily detected to the sulfate-containing fractions, IDs 91-95, indicating the presence of the 3-sulfoglucuronyl sequence in these. A striking difference was observed in the number of fractions bound by the two anti-Le^{x}-related antibodies: anti-L5 bound to eight of the neutral fractions, IDs 68-70, 72-76, and to one of the sialic acid-containing fractions, ID 83, whereas anti-SSEA-1 bound only to fraction ID 76. This indicated that there are two distinct Le^{x}-related oligosaccharide populations among the O-glycans.

Deconvolution of Lewis^{x} Bearing O-glycan Fractions IDs 75 (r4) and 76 (r5).

Insight was gained into the structural basis of the wide range of binding of anti-L5 and the restricted binding of anti-SSEA-1, through deconvolution steps: (i) TLC of the L5-positive/SSEA-1-positive fraction, ID 76 (r5) shown in FIG. 4, and the neighboring SSEA-1-negative/L5-positive fraction, ID 75 (r4), FIG. 4, (ii) probing the chromatograms with the two antibodies, and (iii) mass spectrometric analyses in situ. With anti-SSEA-1, binding was detected only to one NGL component in fraction ID 76, but not to those in ID 75 despite a heavier loading of this fraction (FIG. 5A). With anti-L5, clear binding signals were observed not only with the SSEA-1-positive component designated L⁺/S⁺, but also with several components in ID 75, of which the main component was designated L⁺/S⁻. Mass spectra acquired from the TLC surface revealed the difference in sequence (FIG. 5B). The L⁺/S⁻ component gave two molecular ions at m/z 1494 and 1464, indicating that it is a trisaccharide, Hex-HexNAc(Fuc)-, linked to the lipid ADHP through a three and a two carbon fragment, respectively, generated by periodate oxidation²⁰. The trisaccharide sequence was corroborated by sequence ions of the major component ([M−H]⁻ m/z 1494) at m/z 1348 and 1332. The L⁺/S⁺ contained a single component with a molecular ion at m/z 2224, contains a heptasaccharide sequence linked to ADHP through a three carbon fragment, Hex-HexNAc(Fuc)-Hex-HexNAc-Hex-HexNAc- as indicated by the partial sequence ions m/z 2078, 2062, 1551, 1348 and 1186. Based on earlier work which established Le^{x} rather than Le^{a} specificities of the two antibodies^{24,26,33}, we conclude that the anti-L5 can bind to Le^{x} antigen on a disaccharide or longer backbone, whereas anti-SSEA-1 is stringent in its requirement for a longer, neo-lacto backbone. We infer that the majority of the Le^{x} antigen on O-glycans in the microarray is based on the disaccharide backbone, whereas, Le^{x} based on a neo-lacto backbone is sparse. It is interesting to recall that anti-L5 was originally raised to glycoproteins of the nervous system³⁴, and was shown²⁶ to bind to the short 3′-fucosyl-N-acetyllactosamine sequence capping the trimannosyl core of N-glycans, as well as to the 3′-fucosyl-neo-tetraose sequence, LNFP III.

Conclusions

Numerous approaches have been made in the past to the identification of oligosaccharides interacting with specific proteins. Among them, a solid phase combinatorial synthesis approach for di- and trisaccharides³⁵, and direct colorimetric detections of interactions with sialic acids³⁶. We describe here an effective means of presenting as microarrays, diverse oligosaccharides, not only those of defined sequences, but also complex mixtures (‘libraries’) generated from desired glycoproteins and proteoglycans or an organ, for examining protein-carbohydrate interactions. The technology encompasses the oligosaccharide repertoire of glycosphingolipids; it incorporates chemically synthesized oligosaccharides, and would be particularly suitable for detecting oligosaccharide ligands among soluble oligosaccharides or lipid linked oligosacchride products of combinatorial syntheses. Multivalent display of oligosaccharide ligands is of key importance in carbohydrate-protein interactions as the affinities of oligosaccharide ligands in the monovalent state are generally very low³⁷. Lipid-linked oligosaccharides when immobilized on matrices such as plastic microwells or chromatograms, or displayed on liposomes satisfy very effectively the requirement for multivalent display on account of the close stacking of their lipid moieties³⁸⁻⁴¹. Our present results show that there is excellent presentation of the lipid-linked oligosaccharides also on nitrocellulose, such that the loadings of carbohydrate material are 10-30 times lower than the optimal loadings for chromatogram binding experiments⁴². With more sensitive detection systems for protein binding, there will be further lowering of requirements for carbohydrate loading. The microarray format maximizes capacity; with the existing equipment, on a typical coated slide, 20×50 mm, 1000 spots could be printed. The number of spots per unit area would be substantially increased with robotic microarrayer equipment.

In the present exploratory study, we have illustrated the potential of the oligosaccharide microarrays in the detection and assignment of the specificities of protein-glycosaminoglycan interactions, taking as examples an antiserum, a monoclonal antibody, the cytokine INFγ, and the chemokine RANTES. Among the novel data generated are the interactions of INFγ and RANTES, not only with oligosaccharides of chondroitin sulfates, but also with other sulfated sequences: the HNK-1 sequence characteristic of natural killer cells, and the sulfated sequences of the Lewis^{a} and Lewis^{x} series, which are known to occur on epithelial cells. These findings may be novel clues to the tissue targeting of INFγ and RANTES, and they open the way to the detailed assignment of the motifs recognized by these effector proteins of the immune system.

The observations with the brain-derived O-glycan microarray highlight the power of NGL technology to probe heterogeneous glycan populations. A relatively simple deconvolution approach sufficed with the two oligosaccharide fractions, ID 75 and 76, investigated here. For more complex glycan populations, more extensive fractionations would be required in the construction of the primary arrays, and also the fabrication of sub-arrays or daughter arrays from ligand-positive parent spots. On the one hand, sequence-specific antibodies, such as those used here, could be applied for high-throughput and high sensitivity surveying for the presence of defined oligosaccharide sequences in populations of unknown oligosaccharides. On the other hand, whole cell- or organ-derived glyco-arrays could be used for pinpointing novel ligands for carbohydrate-binding proteins, and for discoveries of novel carbohydrate-binding proteins.

In sum, the novelty of our oligosaccharide array strategy is the unprecedented scope. It has the potential to survey an entire glycome for specific recognition motifs for carbohydrate-binding proteins. Once detected among the great diversity of oligosaccharides, they can be characterized. This is of key importance to understanding protein-oligosaccharide interactions in biological systems, and differs from previously described carbohydrate arrays that have focused on macromolecular polysaccharides or monosaccharides. Although an ideal would be to array all oligosaccharide sequences in a glycome after characterization, this is not achievable currently, and the deconvolution aspects of our strategy permit efforts to be focused on the characterization of the ligand-positive oligosaccharides. In conjunction with advanced protein expression systems, mass spectrometry and bioinformatics, the principle of constructing oligosaccharide arrays from desired sources could form the foundation for surveys to identify oligosaccharide-recognizing proteins in the proteome, and to map the repertoire of complementary recognition structures in the glycome.

Technical Note

As discussed, our strategy has the potential to survey for ligand status oligosaccharide sequences whether known or unknown in an entire glycome, namely to cover what is virtually an unfathomable number, taking into account the diverse backbone and peripheral regions of the sugar chains of glycoproteins and glycolipids and the non-carbohydrate substituents (e.g. sulfation and phosphorylation) that may occur. Below is an account of the range of oligosacchrides which can be sourced for generation of NGLs.

NGLs are readily prepared by reductive-amination of the reducing oligosaccharides with an amino lipid ^{14,43}. These may be (a) naturally occurring free oligosaccharides or chemically synthesized, (b) N-glycans released enzymatically⁴⁴ or by hydrazinolysis⁴⁵, (c) O-glycans released by hydrazinolysis⁴⁶, mild alkaline hydrolysis¹⁸ or 1-glycanase enzyme⁴⁷, (d) the carbohydrate chains of glycolipids released by endo-ceramidase digestion⁴⁸, (e) glycosaminoglycan fragments released by lyases⁴⁹ or nitrous acid treatment⁵⁰, and (f) oligosaccharides obtained by chemical fragmentation of diverse polysaccharides of microbial and plant origins. A limitation of the open chain form of the reducing terminal residue after reductive-amination, and hence loss of the ring form of this residue, is that this may affect ligand activity if it is part of the recognition motif. However, most of the known carbohydrate-recognizing proteins interact with peripheral or backbone sequences of oligosaccharides and in all these cases the chain-opened monosaccharide residue simply acts as a flexible linker between oligosaccharide and lipid. Further development is underway of the technology to preserve the closed ring structure.

The usual method of releasing O-glycans by alkaline borohydride treatment⁵¹ yields reduced oligosaccharide alditols, which cannot be conjugated to the aminolipid directly. A mild periodate oxidation^{21,52,53} was developed to specifically cleave the open-chain reduced-end monosaccharides, without degrading the diols in the saccharide ring, and leaving intact the side chains of the majority of sialic acid residues. In the case of O-glycans with 3,6-disubstituted GalNAcol at their cores, the oxidation will split the core monosaccharide into two portions. This compromises the integrity of the branched core region, but can be exploited for providing sequence information on the branches at the core.

Experimental Protocol

Reducing Oligosaccharides Derived from Glycoproteins, Proteoglycans, Polysaccharides, and Human Milk or by Chemical Synthesis.

Two to 20mer fractions (IDs 22-27, 31-54) were prepared⁵⁴ from chondroitin sulfates A, B and C. An octasaccharide fraction (ID 29) was isolated from porcine intestinal heparan sulfate, HS-1²², after partial depolymerization by nitrous acid (by courtesy of Drs Camilla Westling and Ulf Lindahl, Uppsala Univerisity, Sweden). O-glycan fractions, BSM-N6 (ID3, containing predominantly neutral difucosylated oligosaccharides), BSM-A4 (ID5, mainly sialyl non-fucosylated trisaccharides) and BSM-A6 (ID 4, sialyl, monofucosyl hexasaccharides) were obtained from bovine submaxillary mucin¹⁸. The following oligosaccharides have been described previously: keratan sulfate (KS) hexasaccharide, C4U⁵⁵ (ID 30); penta-mannose phosphate (Man5-phosphate, ID 21) from Hansenula holstii⁵⁶; 3′-sialyl-Lewis^{a} pentasaccharide (designated 3′-SA-Le^{a}-5, ID 13) from human milk, the chemically synthesized oligosaccharides: 3′-sialyl-Lewis^{x} pentasaccharide (3′-SA-Le^{x}-5, ID 14)⁵⁷; 3′-sulfo-Lewis^{a} pentasaccharide (3′-SU-Le^{a}-5, ID 15); 3′-sulfo-Lewis^{x} pentasaccharide (3′-SU-Le^{x}-5, ID 16); 3′,6-sulfo-Lewis^{x} (3′,6-SU-Le^{x}-5, ID 17)⁵⁸⁻⁶⁰. The following oligosaccharides were from commercial sources: lacto-N-tetraose (LNT, ID 6); lacto-N-neo-tetraose (LNnT, ID 7); lacto-N-fucopentaoses I, II, and III (LNFP I, II and III, IDs 8-10, respectively); lacto-N-difucohexaose-I (LNDFH I, ID 11); lacto-N-neo-difucohexaose I (LNnDFH I, ID 12) and heparan sulfate/heparin disaccharide, IS (HS/Hep 2mer, ID 28); the high mannose (Man6, ID 1) N-glycan and the sialyl-biantennary N-glycan (ID 2).

Brain-Derived O-Glycan Alditol Fractions.

O-glycan alditol fractions were obtained after alkaline borohydride treatment of total pronase glycopeptides derived from rabbit brain glycoproteins¹⁹. Those included in the present study are tri- to octasaccharide fractions, separated into neutral (IDs 67-76), sialylated (IDs 77-90) and sulfated (IDs 91-95) subfractions by anion-exchange chromatography²⁰ before further fractionation by normal-phase HPLC^{19,20}.

Lipid-Linked Oligosaccharides.

Oligosaccharides were converted to NGLs by conjugating to 1,2-dihexadecyl-sn-glycero-3-phosphoethanolamine (DHPE) directly (reducing oligosaccharides), or after mild periodate oxidation (reduced oligosaccharides) as described^{21,43}. The NGLs of reduced oligosaccharide fractions from the brain glycopeptides were prepared using the fluorescent lipid, N-aminoacetyl-N-(9-anthracenylmethyl)-1,2-dihexadecyl-sn-glycero-3-phosphoethanolamine (ADHP)¹⁵. The following glycolipids, described previously, were chemically synthesized and contain the following sequences: 6′-sulfo-3′-sialyl-Le^{x} pentasaccharide (6′-SU-3′-SA-Le^{x}-5, ID 19), 6-sulfo-3′-sialy-Le^{x} pentasaccharide (6-SU-3′-SA-Le^{x}-5, ID 18)^{61,62}; 3-sulfoglucuronyl-neo-tetraose, HNK-1 antigen⁶³ (ID 20). The fluorescent NGLs were visualized directly under 254 nm UV light, and the non-fluorescent NGLs and glycolipids were visualized under 366 nm UV light following primulin staining

Mass Spectrometry.

Liquid secondary-ion mass spectrometry was carried out on a VG Analytical ZAB2-E mass spectrometer; spectra were acquired in negative-ion mode as described⁶⁴.

Antibodies and Recombinant Proteins.

Murine anti-SSEA-1 (ascites)²⁴, purchased from Developmental Studies Hybridoma Bank, University of Iowa, and rat anti-L5 (purified from culture supernatant)²⁶, provided by Dr Melitta Shachner (Institute for Biosynthesis of Neural Structures, Hamburg, Germany) are monoclonal antibodies that recognize Lewis^{x}-containing oligosaccharides. Murine monoclonal antibody C-14 (culture supernatant), which recognizes the Le^{y} sequence²⁷ was provided by Dr. Tina Parsons (City Hospital, Nottingham, UK). Monoclonal antibody, HNK-1 (isolated from culture supernatants) recognizes the 3-sulfoglucuronyl-lacto-N-neo-lactosaminyl sequence^{19,23}. Murine monoclonal antibody that recognizes chondroitin sulfates A and C, CS-56 (ascites)²⁸ and rabbit antiserum to chondroitinase ABC-treated chondroitin sulfates⁶⁵, which we designate α-CSΔ were from Bio-Genesis. Recombinant, soluble E-selectin-IgM chimera⁶⁶ was provided by Dr John Lowe (University of Michigan, Ann Arbor, Mich.), and recombinant soluble L-selectin IgG chimera was isolated from the culture supernatant of transfected Chinese hamster ovary cells⁵⁵ that were provided by Dr Gray Shaw (Genetics Institute, Cambridge, Mass.). The recombinant human cytokine, interferon γ (INFγ) and the chemokine RANTES expressed in Escherichia coli were from Sigma and R&D systems, respectively.

Immobilization of Lipid-Linked Oligosaccharides.

The lipid-linked oligosaccharides (in chloroform:methanol:water, 25:25:8 v/v) were applied by jet spray with a sample applicator (Linomat IV, Camag, Switzerland) as 2 mm bands for ‘miniarray’, or as 3001m spots for ‘microarray’ format, onto 0.45 μm nitrocellulose or 0.2 μm PVDF membranes (both from Bio-Rad, Hemel Hempstead, UK), or aluminium-backed silica gel plates (Merck). When fluorescent NGLs were applied onto the membranes, and examined directly under UV light, 1-2 pmol was clearly visible above the background on the PVDF, whereas on nitrocellulose, 5 pmol was required due to a high fluorescent background. For detection, therefore, the fluorescent NGLs were printed onto PVDF membranes; the non-fluorescent lipid-linked oligosaccharides were printed onto silica gel plates and detected with primulin stain.

To assess the retention of lipid-linked oligosaccharides printed on the nitrocellulose and PVDF membranes, the fluorescent NGLs of LNFP III and CSC 16mer were selected as examples of neutral and highly acidic NGLs, respectively, and applied as 2 mm bands onto duplicate sheets. One sheet was subjected to repeated washing procedures analogous to those in the carbohydrate-binding experiments; with the PVDF membrane, a pre-wetting step, using acetonitrile:water, 30:70 (v/v) was included before washing. The bands were quantified by scanning for fluorescence with a Shimadzu CS 9000 scanning densitometer. When the amounts applied were in the range used for carbohydrate-binding experiments (10 pmol), the neutral NGL was retained equally well (about 50% retained) on nitrocellulose and PVDF membranes after washing procedures. The retention of the acidic NGL on the nitrocellulose was also about 50%, but on the PVDF membrane little was retained (10% or less).

Carbohydrate-Binding Experiments with Lipid-Linked Oligosaccharide Probes Printed on Membranes.

Experiments were carried out at ambient temperature unless stated otherwise. The dilutions of the antibodies used were those recommended by the manufacturers or providers. Concentrations where known are indicated. For antibody-binding experiments, the membranes were ‘blocked’ for 1 h with 3% (w/v) bovine serum albumin (BSA) in phosphate buffered saline (10 mM phosphate buffer containing 150 mM NaCl) pH 7.4 (PBS), and overlaid with for 2 h with the antibodies diluted in phosphate buffered saline containing 3% BSA (PBS/BSA). The rodent antibodies were diluted as follows in PBS/BSA: anti-L5 at 1:50, anti-SSEA-1, 1:100, anti-HNK-1, 1:500 and CS-56, 1:200, and the rabbit anti-chondroitin sulfate serum α-CS, at 1:1600 dilution. Membranes were washed four times with PBS, and overlaid for 1 h with anti-mouse immunoglobulins (Dako) or anti-rabbit IgG conjugated to horseradish peroxidase (HRP, Sigma) at 5 μg/ml in PBS/BSA. Antibody-binding was detected by development with FAST 3, 3′-diaminobenzidine (DAB-FAST) reagent (Sigma) according to the manufacturer's instructions.

Selectin-binding experiments were carried out as above except that the membranes were ‘blocked’ 1% (w/v) casein in 10 mM Tris-HCl buffer pH 7.4 containing 50 mM CaCl₂ and 150 mM NaCl (TBS/Ca) and overlaid for 2 h with L-selectin (1 μg/ml pre-complexed⁴² with biotinylated anti-human IgG from Vector), or E-selectin (at 1 μg/ml) in TBS/Ca. Binding of L-selectin was detected by overlaying with HRP-conjugated streptavidin (Sigma), 10 μg/ml, in TBS/Ca containing 1% casein. Binding of E-selectin was detected by overlaying with biotinylated anti-human IgM (Sigma), 5 μg/ml, followed by the HRP-conjugated streptavidin.

For cytokine/chemokine-binding experiments, the membranes were blocked for 1 h with 3% (w/v) BSA in 10 mM Tris-HCl buffer containing 150 mM NaCl, 2 mM CaCl₂ and 0.8 mM MgCl₂ (TNCM), overlaid with INFγ or RANTES at 1 μg per ml diluted in TNCM/BSA, incubated for 16 h at 4° C.

The membranes were washed four times, for 30 min with 10 mM Tris-HCl buffer containing 20 mM NaCl, 5 mM CaCl₂ and 2 mM MgCl₂ (TNCM-L) to minimize elution of the bound proteins³¹. Binding was detected by overlaying for 2 h with a murine IgG₁ monoclonal antibody to human INFγ (Pierce) or a murine IgG₁ monoclonal antibody to human RANTES (R & D Systems) both at 1 μg/ml in TNCM-L containing 3% BSA, followed by overlay with HRP-conjugated anti-mouse immunoglobulins as above. Carbohydrate-binding experiments with NGLs resolved by TLC were performed as described¹².

### REFERENCES

1. Carbohydrates and Glycobiology. Science 291, 2337-2378 (2001).

2. Helenius, A. & Aebi, M. Intracellular functions of N-linked glycans. Science 291, 2364-2369 (2001).

3. Feizi, T. Progress in deciphering the information content of the ‘glycome’—a crescendo in the closing years of the millennium. Glycoconj. J. 17, 553-565 (2000).

4. Crocker, P. R. & Varki, A. Siglecs in the immune system. Immunology 103, 137-145 (2001).

5. Karlsson K. A. The human gastric colonizer Helicobacter pylori: a challenge for host-parasite glycobiology. Glycobiology 10, 761-771 (2000).

6. Kiessling, L. L. & Cairo, C. W. Hitting the sweet spot. Nat. Biotechnol. 20, 234-235 (2002).

7. Schena, M., Shalon, D., Davis, R. W. & Brown, P. O. Quantitative monitoring of gene expression patterns with a complementary DNA microarray. Science 270, 467-470 (1995).

8. Mitchell, P. A perspective on protein microarrays. Nat. Biotechnol. 20, 225-229 (2002).

9. Wang, D., Liu, S., Trummer, B. J., Deng, C. & Wang, A. Carbohydrate microarrays for the recognition of cross-reactive molecular markers of microbes and host cells. Nat. Biotechnol. 20, 275-281 (2002).

10. Houseman, B. T. & Mrksich, M. Carbohydrate arrays for the evaluation of protein binding and enzymatic modification. Chem. Biol. 9, 443-454 (2002).

11. Tang, P. W., Gooi, H. C., Hardy, M., Lee, Y. C. & Feizi, T. Novel approach to the study of the antigenicities and receptor functions of carbohydrate chains of glycoproteins. Biochem. Biophys. Res. Commun. 132, 474-480 (1985).

12. Stoll, M. S., Mizuochi, T., Childs, R. A. & Feizi, T. Improved procedure for the construction of neoglycolipids having antigenic and lectin-binding activities from reducing oligosaccharides. Biochem. J. 256, 661-664 (1988).

13. Lawson, A. M. et al. High-sensitivity structural analyses of oligosaccharide probes (neoglycolipids) by liquid-secondary-ion mass spectrometry. Carbohydr. Res. 200, 47-57 (1990).

14. Feizi, T., Stoll, M. S., Yuen, C.-T., Chai, W. & Lawson, A. M. Neoglycolipids: probes of oligosaccharide structure, antigenicity and function. Methods Enzymol. 230, 484-519 (1994).

15. Stoll M S et al. Fluorescent neoglycolipids: improved probes for oligosaccharide ligand discovery. Eur. J. Biochem. 267, 1795-1804 (2000).

16. Feizi, T. Carbohydrate Bioengineering: Interdisciplinary Approaches. Teeri, T. T., Svensson, B., Gilbert, H. J. & Feizi, T. (eds.), pp. 186-193 (The Royal Society of Chemistry,2002).

17. Yuen, C.-T. et al. Novel sulfated ligands for the cell adhesion molecule E-selectin revealed by the neoglycolipid technology among O-linked oligosaccharides on an ovarian cystadenoma glycoprotein. Biochemistry 31, 9126-9131 (1992).

18. Chai, W., Feizi, T., Yuen, C.-T. & Lawson, A. M. Nonreductive release of O-linked oligosaccharides from mucin glycoproteins for structure/function assignments as neoglycolipids: application in the detection of novel ligands for E-selectin. Glycobiology 7, 861-872 (1997).

19. Yuen, C.-T. et al. Brain contains HNK-1 immunoreactive O-glycans of the sulfoglucuronyl lactosamine series that terminate in 2-linked or 2,6-linked hexose (mannose). J. Biol. Chem. 272, 8924-8931 (1997).

20. Chai, W. et al. High prevalence of 2-mono- and 2,6-di-substituted Manol-terminating sequences among O-glycans released from brain glycopeptides by reductive alkaline hydrolysis. Eur. J. Biochem. 263, 879-888 (1999).

21. Chai, W., Yuen, C.-T., Feizi, T. & Lawson, A. M. Core-branching pattern and sequence analysis of mannitol-terminating oligosaccharides by neoglycolipid technology. Anal. Biochem. 270, 314-322 (1999).

22. Leteux, C., Chai, W., Nagai, K., Lawson, A. M. & and Feizi, T. 10E4 Antigen of scrapie lesions contains an unusual nonsulfated heparan motif. J. Biol. Chem. 276, 12539-12545 (2001).

23. Chou, D. K. H. et al. Structure of sulfated glucuronyl glycolipids in the nervous system reacting with HNK-1 antibody and some IgMparaproteins in neuropathy. J. Biol. Chem. 261, 11717-11725 (1986).

24. Gooi, H. C. et al. Stage specific embryonic antigen SSEA-1 involves a1-3 fucosylated type 2 blood group chains. Nature 292, 156-158 (1981).

25. Gooi, H. C. et al. Marker of peripheral blood granulocytes and monocytes of man recognized by two monoclonal antibodies VEP8 and VEP9 involves the trisaccharide 3-fucosyl-N-acetyllactosamine. Eur. J. Immunol. 13, 306-312 (1983).

26. Streit, A. et al. The Lex, carbohydrate sequence is recognized by antibody to L5, a functional antigen in early neural development. J. Neurochem. 66, 834-844 (1996).

27. Brown, A. et al. A monoclonal antibody against human colonic adenoma recognizes difucosylated Type-2 blood group chains. Biosci. Reps. 3, 163-170 (1983).

28. Avnur, Z. & Geiger, B. Immunocytochemical localization of native chondroitin-sulfate in tissues and cultured cells using specific monoclonal antibody. Cell 38, 811-822 (1984).

29. Rosen, S. D. & Bertozzi, C. R. Leukocyte adhesion: Two selectins converge on sulphate. Curr. Biol. 6, 261-264 (1996).

30. Feizi, T. Mammalian Carbohydrate Recognition Systems. Results and Problems in Cell Differentiation. Paul R. Crocker (ed.), pp. 201-223 (Springer-Verlag Berlin Heidelberg, 2001).

31. Hurt-Camejo, E. et al. CD44, a cell surface chondroitin sulfate proteoglycan, mediates binding of interferon-gamma and some of its biological effects on human vascular smooth muscle cells. J. Biol. Chem. 274, 18957-18964 (1999).

32. Hirose, J., Kawashima, H., Yoshie, O., Tashiro, K. & Miyasaka, M. Versican interacts with chemokines and modulates cellular responses. J. Biol. Chem. 276, 5228-5234 (2001).

33. Hounsell, E. F. et al. Structural analysis of hexa- to octa-saccharide fractions isolated from sheep gastric-glycoproteins having blood-group I and i activities. Carbohydr. Res. 90, 283-307 (1981).

34. Roberts, C., Platt, N., Streit, A., Schachner, M. & Stem, C. D. The L5 epitope: an early marker for neural induction in the chick embryo and its involvement in inductive interactions. Development 112, 959-970 (1991).

35. Liang, R. et al. Parallel synthesis and screening of a solid phase carbohydrate library. Science 274, 1520-1522 (1996).

36. Charych, D. H., Nagy, J. O., Spevak, W. & Bednarski, M. D. Direct colorimetric detection of a receptor-ligand interaction by a polymerized bilayer assembly. Science 261, 585-588 (1993).

37. Crocker, P. R. & Feizi, T. Carbohydrate recognition systems: functional triads in cell-cell interactions. Curr. Opin. Struct. Biol. 6, 679-691 (1996).

38. Ghosh, P., Bachhawat, B. K. & Surolia, A. Synthetic glycolipids: interaction with galactose-binding lectin and hepatic cells. Arch. Biochem. Biophys. 206, 454-457 (1981).

39. Surolia, A., Bachhawat, B. K. & Podder, S. K. Interaction between lectin from Ricinus communis and liposomes containing gangliosides. Nature 257, 802-804 (1975).

40. Galustian, C. et al. Synergistic interactions of the two classes of ligand, sialyl-Lewisa/x fuco-oligosaccharides and short sulpho-motifs, with the P- and L-selectins: implications for therapeutic inhibitor designs. Immunology 105, 350-359 (2002).

41. Srinivas, O., Mitra, N., Surolia, A. & Jayaraman, N. Photoswitchable Multivalent Sugar Ligands: Synthesis, Isomerization, and Lectin Binding Studies of Azobenzeneminus signGlycopyranoside Derivatives. J. Am. Chem. Soc. 124, 2124-2125 (2002).

42. Galustian, C. et al. Valency dependent patterns of reactivity of human L-selectin towards sialyl and sulfated oligosaccharides of Lea and Lex types: Relevance to anti-adhesion therapeutics. Biochemistry 36, 5260-5266 (1997).

43. Stoll, M. S. & Feizi, T. A laboratory guide to glycoconjugate analysis. Jackson, P. & Gallagher, J. T. (eds.), pp. 329-348 (Birkhauser Verlag A G, Basel, Switzerland, 1997).

44. Plummer, T. H., Jr., Elder, J. H., Alexander, S., Phelan, A. W. & Tarentino, A. L. Demonstration of peptide:N-glycosidase F activity in endo-beta-N-acetylglucosaminidase F preparations. J. Biol. Chem. 259, 10700-10704 (1984).

45. Takasaki, S., Mizuochi, T. & Kobata, A. Hydrazinolysis of asparagine-linked sugar chains to rpoduce free oligosaccharides. Methods Enzymol 83, 263-268 (1982).

46. Patel, T. et al. Use of hydrazine to release in intact and unreduced form both N- and O-linked oligosaccharides from glycoproteins. Biochemistry 32, 679-693 (1993).

47. Sutherland, D. R., Abdullah, K. M., Cyopick, P. & Mellors, A. Cleavage of the cell-surface O-sialoglycoproteins CD34, CD43, CD44, and CD45 by a novel glycoprotease from Pasteurella haemolytica. J. Immunol. 148, 1458-1464 (1992).

48. Osanai, T. et al. Two families of murine carbohydrate ligands for E-selectin. Biochem. Biophys. Res. Commun. 218, 610-615 (1996).

49. Chai, W., Luo, J., Lim, C. K. & Lawson, A. M. Characterization of heparin oligosaccharide mixtures as ammonium salts using electrospray mass spectrometry. Anal. Chem. 70, 2060-2066 (1998).

50. Shively, J. E. & Conrad, H. E. Formation of anhydrosugars in the chemical depolymerization of heparin. Biochemistry 15, 3932-3942 (1976).

51. Iyer, R. N. & Carlson, D. M. Alkaline borohydride degradation of blood group H substance. Arch. Biochem. Biophys. 142, 101-105 (1971).

52. Stoll, M. S., Hounsell, E. F., Lawson, A. M., Chai, W. & Feizi, T. Microscale sequencing of O-linked oligosaccharides using mild periodate oxidation of alditols, coupling to phospholipid and TLC-MS analysis of the resulting neoglycolipids. Eur. J. Biochem. 189, 499-507 (1990).

53. Chai, W., Stoll, M. S., Cashmore, G. C. & Lawson, A. M. Specificity of mild periodate oxidation of oligosaccharide-alditol relevance to the analysis of the core-branching pattern of O-linked glycoprotein oligosaccharides. Carbohydr. Res. 239, 107-115 (1993).

54. Chai, W., Kogelberg, H. & Lawson, A. M. Generation and structural characterization of a range of unmodified chondroitin sulfate oligosaccharide fragments. Anal. Biochem. 237, 88-102 (1996).

55. Galustian, C. et al. L-selectin interactions with novel mono- and multisulfated Lewisx sequences in comparison with the potent ligand 3′-sulfatedLewisa. J. Biol. Chem. 274, 18213-18217 (1999).

56. Loveless, R. W., Floyd-O'Sullivan, G., Raynes, J. G. & Feizi, T. Human serum amyloid P is a multispecific adhesive protein whose ligands include 6-phosphorylated mannose and the 3-sulphated saccharides galactose, N-acetylgalactosamine and glucuronic acid. EMBO J. 11, 813-819 (1992).

57. Hasegawa, A., Ando, T., Kameyama, A. & Kiso, M. Stereocontrolled synthesis of sialyl Lewis X ceramide consisting of pentasaccharide recognized selectin family. Carbohydr. Res. 230, cl-cS (1992).

58. Lubineau, A., Le-Gallic, J. & Lemoine, R. First synthesis of the 3′-sulfated Lewis(a) pentasaccharide, the most potent human E-selectin ligand so far. Bioorg. Med. Chem. 2, 1143-1151 (1994).

59. Lubineau, A., Auge, C., Le Goff, N. & Le Narvor, C. Chemoenzymatic synthesis of a 31V,61II-disulfated Lewis(x) pentasaccharide, a candidate ligand for human L-selectin. Carbohydr. Res. 305, 501-509 (1997).

60. Lubineau, A., Alais, J. & Lemoine, R. Synthesis of 3′- and 6′-monosulfated and 3′,6′-disulfated Lewis x pentasaccharide, candidate ligands for human L-selectin. J. Carbohydr. Chem. 19, 151-169 (2000).

61. Komba, S., Ishida, H., Kiso, M. & Hasegawa, A. Synthesis and biological activities of three sulfated sialyl Le(x) ganglioside analogues for clarifying the real carbohydrate ligand structure of L-selection. Bioorg. Med. Chem. 4, 1833-1847 (1996).

62. Galustian, C. et al. Sialyl-Lewisx sequence 6-O-sulfated at N-acetylglucosamine rather than at galactose is the preferred ligand for L-selectin and de-N-acetylation of the sialic acid enhances the binding strength. Biochem. Biophys. Res. Comm. 240, 748-751 (1997).

63. Isogai, Y., Kawase, T., Ishida, H., Kiso, M. & Hasegawa, A. Total synthesis of sulfated glucuronyl paraglobosides. J. Carbohydr. Chem. 15, 1001-1023 (1996).

64. Chai, W., Cashmore, G. C., Carruthers, R. A., Stoll, M. S. & Lawson, A. M. Optimal procedure for combined high-performance thin-layer chromatography/high-sensitivity liquid secondary ion mass spectrometry. Biol. Mass Spectrom. 20, 169-178 (1991).

65. Bertolotto, A., Palmucci, L., Gagliano, A., Mongini, T. & Tarone, G. Immunohistochemical localization of chondroitin sulfate in normal and pathological human muscle. J. Neurol. Sci. 73, 233-244 (1986).

66. Smith, P. L. et al. Expression of the a(1-3)fucosyltransferase Fuc-TVII in lymphoid aggregate high endothelial venules correlates with expression of L-selectin ligands. J. Biol. Chem. 271, 8250-8259 (1996).

TABLE 1
Designations and locations of the oligosaccharides investigated.

| ID^{a} | Oligosaccharides^{b} | Type^{c} | Location | Sequences^{d} |
|---|---|---|---|---|
| 1 | N-glycan Man6 | N | a1 | Maα-6(Maα3)Maα-6(Maα-2Maα3)Maβ-4GcNβ-4GcN |
| 2 | N-glycan | N | a2 | SAα-6Gaβ-4GcNβ-2Maβ-6(SAα-6Gaβ-4GcNβ-2Maβ- |
| | biantennary | | | 3)Maα-4GcNβ-4GcN |
| 3 | O-glycan | N | a3 | (Fu)₂.(Hx)₂.(HxN)₄ (BSM fraction N6) |
| | fucosylated | | | |
| 4 | O-glycan SA- | N | a4 | SA.Fu.Hx.(HxN)₃ (BSM fraction A6) |
| | fucosyl | | | |
| 5 | O-glycan sialyl | N | a5 | SA.(HxN)₂ (BSM fraction A4) |
| 6 | LNT | N | b1 | Gaβ-3GcNβ-3Gaβ-4Gc |
| 7 | LNnT | N | b2 | Gaβ-4GcNβ-3Gaβ-4Gc |
| 8 | H (LNFP I) | N | b3 | Fuα-2Gaβ-3GcNβ-3Gaβ-4Gc |
| 9 | Le^{a} (LNFP II) | N | b4 | Gaβ-(Fuα-4)3GcNβ-3Gaβ-4Gc |
| 10 | Le^{x} (LNFP III) | N | b5 | Gaβ-(Fuα-3)4GcNβ-3Gaβ-4Gc |
| 11 | Le^{b} (LNDFH I) | N | c1 | Fuα-2Gaβ-(Fuα-4)3GcNβ-3Gaβ-4Gc |
| 12 | Le^{y} (LNnDFH I) | N | c2 | Fuα-2Gaβ-(Fuα-3)4GcNβ-3Gaβ-4Gc |
| 13 | 3′-SA-Le^{a}-5 | S | c3 | SAα-3Gaβ-3(Fuα-4)GcNβ-3Gaβ-4Gc |
| 14 | 3′-SA-Le^{x}-5 | S | c4 | SAα-3Gaβ-4(Fuα-3)GcNβ-3Gaβ-4Gc |
| 15 | 3′-SU-Le^{a}-5 | S | c5 | Ga(3SU)β-3(Fuα-4)GcNβ-3Gaβ-4Gc |
| 16 | 3′-SU-Le^{x}-5 | S | d1 | Ga(3SU)β-4(Fuα-3)GcNβ-3Gaβ-4Gc |
| 17 | 3′,6-SU-Le^{x}-5 | S | d2 | Ga(3SU)β-4(Fuα-3)GcN(6SU)β-3Gaβ-4Gc |
| 18 | 6-SU-3′-SA-Le^{x} | G | d3 | SAα-3Gaβ-3(Fuα-4)GcN(6SU)β-3Gaβ-4Gc |
| 19 | 6′-SU-3′-SA-Le^{x}5 | G | d4 | SAα-3Ga(6SU)β-3(Fuα-4)GcNβ-3Gaβ-4Gc |
| 20 | HNK-1 | G | d5 | GcA(3SU)β-3Gaβ-4GcNβ-3Gaβ-4Gc |
| 21 | Man5-phosphate | N | e1 | Ma(6PA)α-3Maα-3Maα-3Maα-2Ma |
| 22 | CSA 2mer | N | e2 | ΔUA-3GaN(4SU) |
| 23 | CSA 14mer | N | e3 | ΔUA-[3GaN(4SU)β-4GcAβ]₆-3GaN(4SU) |
| 24 | CSB 2mer | N | e4 | ΔUA-3GaN(4SU) |
| 25 | CSB 14mer | N | e5 | ΔUA-[3GaN(4SU)β-4IdAα]₆-3GaN(4SU) |
| 26 | CSC 2mer | N | f1 | ΔUA-3GaN(6SU) |
| 27 | CSC 14mer | N | f2 | ΔUA-[3GaN(6SU)β-4IdAα]₆-3GaN(6SU) |
| 28 | HS/HEP 2mer | N | f3 | ΔUA(2SU)-4GcNSU(6SU) |
| 29 | HS/HEP 8mer | N | f4 | UA-[GcN-UA]₃-anMa |
| 30 | KS 4mer (C4U) | N | f5 | SAα-3Gaβ-4(Fuα-3)(SU-6)GcNβ-3(SU-6)Gaβ-4(SU-6)GcN |
| 22 | CSA 2mer | N | g1 | ΔUA-3GaN(4SU) |
| 31 | CSA 4mer | N | g2 | ΔUA-3GaN(4SU)β-4GcAβ-3GaN(4SU) |
| 32 | CSA 6mer | N | g3 | ΔUA-[3GaN(4SU)β-4GcAβ]₂-3GaN(4SU) |
| 33 | CSA 8mer | N | g4 | ΔUA-[3GaN(4SU)β-4GcAβ]₃-3GaN(4SU) |
| 34 | CSA 10mer | N | g5 | ΔUA-[3GaN(4SU)β-4GcAβ]₄-3GaN(4SU) |
| 35 | CSA 12mer | N | g6 | ΔUA-[3GaN(4SU)β-4GcAβ]₅-3GaN(4SU) |
| 23 | CSA 14mer | N | h1 | ΔUA-[3GaN(4SU)β-4GcAβ]₆-3GaN(4SU) |
| 36 | CSA 16mer | N | h2 | ΔUA-[3GaN(4SU)β-4GcAβ]₇-3GaN(4SU) |
| 37 | CSA 18mer | N | h3 | ΔUA-[3GaN(4SU)β-4GcAβ]₈-3GaN(4SU) |
| 38 | CSA 20mer | N | h4 | ΔUA-[3GaN(4SU)β-4GcAβ]₉-3GaN(4SU) |
| 24 | CSB 2mer | N | i1 | ΔUA-3GaN(4SU) |
| 39 | CSB 4mer | N | i2 | ΔUA-3GaN(4SU)β-4IdAα-3GaN(4SU) |
| 40 | CSB 6mer | N | i3 | ΔUA-[3GaN(4SU)β-4IdAα]₂-3GaN(4SU) |
| 41 | CSB 8mer | N | i4 | ΔUA-[3GaN(4SU)β-4IdAα]₃-3GaN(4SU) |
| 42 | CSB 10mer | N | i5 | ΔUA-[3GaN(4SU)β-4IdAα]₄-3GaN(4SU) |
| 43 | CSB 12mer | N | i6 | ΔUA-[3GaN(4SU)β-4IdAα]₅-3GaN(4SU) |
| 25 | CSB 14mer | N | j1 | ΔUA-[3GaN(4SU)β-4IdAα]₆-3GaN(4SU) |
| 44 | CSB 16mer | N | j2 | ΔUA-[3GaN(4SU)β-4IdAα]₇-3GaN(4SU) |
| 45 | CSB 18mer | N | j3 | ΔUA-[3GaN(4SU)β-4IdAα]₈-3GaN(4SU) |
| 46 | CSB 20mer | N | j4 | ΔUA-[3GaN(4SU)β-4IdAα]₉-3GaN(4SU) |
| 26 | CSC 2mer | N | k1 | ΔUA-3GaN(6SU) |
| 47 | CSC 4mer | N | k2 | ΔUA-3GaN(6SU)β-4GcAβ-3GaN(6SU) |
| 48 | CSC 6mer | N | k3 | ΔUA-[3GaN(6SU)β-4GcAβ]₂-3GaN(6SU) |
| 49 | CSC 8mer | N | k4 | ΔUA-[3GaN(6SU)β-4GcAβ]₃-3GaN(6SU) |
| 50 | CSC 10mer | N | k5 | ΔUA-[3GaN(6SU)β-4GcAβ]₄-3GaN(6SU) |
| 51 | CSC 12mer | N | k6 | ΔUA-[3GaN(6SU)β-4GcAβ]₅-3GaN(6SU) |
| 27 | CSC 14mer | N | l1 | ΔUA-[3GaN(6SU)β-4GcAβ]₆-3GaN(6SU) |
| 52 | CSC 16mer | N | l2 | ΔUA-[3GaN(6SU)β-4GcAβ]₇-3GaN(6SU) |
| 53 | CSC 18mer | N | l3 | ΔUA-[3GaN(6SU)β-4GcAβ]₈-3GaN(6SU) |
| 54 | CSC 20mer | N | l4 | ΔUA-[3GaN(6SU)β-4GcAβ]₉-3GaN(6SU) |
| 55 | CSA 3mer | N | m1 | GaN(4SU)β-4GcAβ-3GaN(4SU) |
| 56 | CSA 5mer | N | m2 | GaN(4SU)β-[4GcAβ-3GaN(4SU)]₂ |
| 57 | CSB 3mer | N | m3 | GaN(4SU)β-4IdAα-3GaN(4SU) |
| 58 | CSB 5mer | N | m4 | GaN(4SU)β-[4IdAα-3GaN(4SU)]₂ |
| 59 | CSC 3mer | N | m5 | GaN(6SU)β-4GcAβ-3GaN(6SU) |
| 60 | CSC 5mer | N | m6 | GaN(6SU)β-[4GcAβ-3GaN(6SU)]₂ |
| 61 | CSC 4mer | N | n1 | GcAβ-3GaN(6SU)β-4GcAβ-3GaN(6SU) |
| 62 | CSC 6mer | N | n2 | GcAβ-[3GaN(6SU)β-4GcAβ]₂-3GaN(6SU) |
| 63 | CSC 8mer | N | n3 | GcAβ-[3GaN(6SU)β-4GcAβ]₃-3GaN(6SU) |
| 64 | CSC 10mer | N | n4 | GcAβ-[3GaN(6SU)β-4GcAβ]₄-3GaN(6SU) |
| 65 | CSC 12mer | N | n5 | GcAβ-[3GaN(6SU)β-4GcAβ]₅-3GaN(6SU) |
| 66 | CSC 14mer | N | n6 | GcAβ-[3GaN(6SU)β-AGcAβ]₆-3GaN(6SU) |
| 67 | RBG 3N2 | R | q1 | neutral |
| 68 | RBG 3N3 | R | q2 | neutral |
| 69 | RBG 3N4 | R | q3 | neutral |
| 70 | RBG 3N5 | R | q4 | neutral |
| 71 | RBG 4N1 | R | q5 | neutral |
| 72 | RBG 4N2 | R | r1 | neutral |
| 73 | RBG 4N5 | R | r2 | neutral |
| 74 | RBG 4N6 | R | r3 | neutral |
| 75 | RBG 5N1 | R | r4 | neutral |
| 76 | RBG 5N2 | R | r5 | neutral |
| 77 | RBG 3A1 | R | s1 | sialyl |
| 78 | RBG 3A4 | R | s2 | sialyl |
| 79 | RBG 3A5 | R | s3 | sialyl |
| 80 | RBG 3A6 | R | s4 | sialyl |
| 81 | RBG 4A4 | R | s5 | sialyl |
| 82 | RBG 4A5 | R | t1 | sialyl |
| 83 | RBG 4A7 | R | t2 | sialyl |
| 84 | RBG 4A8a | R | t3 | sialyl |
| 85 | RBG 4A8b | R | t4 | sialyl |
| 86 | RBG 4A9 | R | t5 | sialyl |
| 87 | RBG 4A10 | R | u1 | sialyl |
| 88 | RBG 4A11 | R | u2 | sialyl |
| 89 | RBG 4A12 | R | u3 | sialyl |
| 90 | RBG 4A15 | R | u4 | sialyl |
| 91 | RBG 2c | R | u5 | sulphated |
| 92 | RBG 2d | R | v1 | sulphated |
| 93 | RBG 2e | R | v2 | sulphated |
| 93 | RBG 3d | R | v3 | sulphated |
| 94 | RBG.3e | R | v4 | sulphated |
| 95 | RBG 3f | R | v5 | Sulphated |

^{a}Oligosaccharides IDs 22-28 are represented both in the ID 1-30 series and (underlined) in the CSA, CSB and CSC series.
^{b}Oligosaccharides IDs 3-5 and the glycosaminoglycan oligosaccharide fractions are heterogeneous. Compositions or sequences of the main components are shown here.
^{c}Abbreviations: N, R and S, natural reducing, natural reduced and synthetic reducing oligosaccharides respectively; G, glycolipids
^{d}Abbreviations: Hx, Hex; HxN, HexNAc; SA, sialic acid; SU, sulfate; UA, hexuronic acid; ΔUA, 4,5-unsaturated hexuronic acid; GcA, glucuronic acid; IdA, iduronic acid; Fu, fucose; Ga, galactose; Gc, glucose; Ma, mannose; anMa, anhydro-mannose; GaN, N-acetylgalactosamine; GcN, N-acetylglucosamine; PA, phosphate; when sequence unknown, composition is given with a dot “.” inserted between monosaccharide residues.

### EXAMPLE 2

### Nitrocellulose Coated Slides as a Support for Carbohydrate Arrays.

Introduction

The invention has been used to generate, to our knowledge, the first arrays of reducing end-tagged carbohydrate molecues with potential to cover the entire range of carbohydrates present in nature (the “glycome”). Immobilisation/fabrication of carbohydrates are through non-covalent interactions rather than covalent bonding to solid matrices.

As will be described in accompanying examples, the arrays can be used for investigations of protein-carbohydrate interactions, but their use can be extended to interactions between carbohydrates with other molecules. Scientifically, they help to answer the question, at molecular level, if a protein recognises carbohydrate and if so what is the ligand. Due to their comprehensiveness in coverage of carbohydrate sequences, it is possible to derive the specificities of such interactions. Commercially, they have applications in, for example, drug development.

The nitrocellulose coated slides used here, FAST Slides, as an example of the support material used in the invention can be obtained from Schleicher and Schuell Bioscience GmbH, Hahnestrasse 3, D-37586 Dassel, Germany.

Materials and Methods

Preparation of Carbohydrate-Containing Molecules.

(1) Oligosaccharides

Free reducing oligosaccharides: any reducing oligosaccharides, such as those isolated from human or animal milk or chemically synthesized.

N-linked glycoprotein oligosaccharides: released by the enzymes peptide-N-(N-acetyl-β-glucosaminyl)asparagine amidase (PNGase F) or endo-β-N-acetylglucosaminidase F (Endo F) or by hydrazinolysis.

O-linked glycoprotein oligosaccharides: released by mild alkaline hydrolysis, by hydrazinolysis.

Natural glycolipids: released by endo-ceramidase.

Proteoglycans or glycosaminoglycans: released from the proteoglycans or the polysaccharides by lyase digestion or nitrous acid degradation, and in the case of hyaluronic acid, also by hydrolase digestion.

Bacterial and plant polysaccharides: released by partial degradation using various chemical methods, including acid or alkaline hydrolysis, acetolysis, Smith degradation.

O-linked mucin-type glycoprotein oligosaccharides: released by reductive alkaline hydrolysis and containing the GalNAcol core.

O-linked mannosyl glycoprotein oligosaccharides: released by reductive alkaline hydrolysis and containing the Mannol core.

Other free oligosaccharide alditols: reduced for other purposes, e.g. for HPLC separation to eliminate the interference caused by the α,β-anomeric forms.

(2) Neoglycolipids (NGLs):

NGLs can be prepared directly from reducing oligosaccharides by reductive-amination with the aminolipid, ADHP or DHPE. NGLs can also be prepared from reduced oligosaccharides by mild periodate oxidation of the open-chain vicinal diol followed by conjugation to the aminolipid, ADHP or DHPE, through reductive-amination.

(3) Other glycoconjugates (glycolipids, glycoproteins, proteoglycans, glycosaminoglycans and polysaccharides): are isolated by established procedure well known to those skilled in the art.

Arraying of Carbohydrate-Containing Molecules.

NGL samples in organic-based solvent mixture (chloroform/methanol/water 25:25:8, by vol), and also polysaccharides, glycosaminoglycans (GAGs) and glycoproteins in water, were arrayed with N₂-assisted jet spray as 1-2 mm bands or 150 μm spots.

A water based solvent/solvent mixture is preferred for arraying. Several formulations of water-based solvent mixtures (containing less volatile organic solvents) are being evaluated for arraying NGL with a pin type microarayers; good results have been obtained with some of the formulations: see FIG. 13 for an example of a microarray of NGLs using a solvent based mixture. However, polysaccharides, glycosaminoglycans (GAGs) and glycoproteins can be easily dissolved in water for arraying.

Imaging of the Results Generated from the Arrays.

Imaging of the NGLs applied was by fluorescence either directly when fluorescent NGLs were used or after primulin staining when non-fluorescent NGLs were used. Otherwise Cy3 dye is included in the carbohydrate solutions as a tracer at a fixed concentration ratio to the carbohydrate, and thus provides a means of monitoring/imaging the fabricated microarrays.

Binding to the arrayed samples was detected using biotinylated antibodies directed to the proteins being investigated, followed by conventional ELISA-DAB colour reaction or by using CyS-labelled streptavidin.

Results and Conclusions

The nitrocellulose coated FAST Slides have been used in this example to examine the sensitivity of detection of protein-carbohydrate interactions, using a selection from our carbohydrate collection: ADHPE derivatives of lacto-N-fucopentaose III (LNFP III) and lacto-N-neotetraose (LNNT); DHPE derivatives of LNFP III, LNNT, 18mer of chondroitin sulphate C (CSC), and 8mer of hyaluronic acid (HA); aminopyridine derivative of LNFP III; and the polysaccharides CSC, Dextran and different preparations of HA. Samples were applied as 1-2 mm bands (larger area for better quantitative analysis and DAB detection). The NGLs used were prepared with an aminolipid 1,2-dihexadecyl-sn-glycero-3-phosphoethanolamine (DHPE) or its fluorescent derivative N-aminoacetyl-N-(9-anthracenylmethyl)-1,2-dihexadecyl-sn-glycero-3-phosphoethanolamine (ADHP).

Detection Sensitivity of NGLs on Nitrocellulose Based Support Material.

Different amounts (1 fmol to 10 pmol) of fluorecent NGLs were applied as 2 mm bands on a nitrocellulose coated FastSlide. The arrangement of the NGLs on the support and quantity used is shown under the data. The NGL used was a Lewis^{X} (Le^{x}) pentasaccharide LNFP III-ADHP. The presence of the NGL on the support material was detected by an anti-Le^{x} antibody (FIG. 7A). A tetrasaccharide LNNT-ADHP (positions 1b and 2b) was used as the negative control, as this molecule not recognised by the anti-Lex antibody.

The binding of the antibody was detected using biotinylated goat anti-mouse immunoglobulins followed by streptavidin-labelled horse radish peroxidase (HRP). Colour development was with fast-diaminobenzidine (DAB). The binding intensity of the antibody to the NGLs was revealed by the DAB colour of each of the bands. The bands were scanned at 550 nm and the colour intensities recorded and plotted for direct comparison (see FIG. 7B).

As can be seen from FIG. 7, the binding of an antibody to the Lewis^{x} pentasaccharide on the nitrocellulose based support can be detected using 400 to 1000 fmoles of NGL. Therefore, the nitrocellulose coated slide is a sensitive support material to use for detecting NGL-molecule binding.

Detection Sensitivity of GA Gs on Nitrocellulose Based Support Material.

Different amounts (5 to 500 ng) of chondroitin sulphate C polysaccharide and its oligosaccharide derived NGL, CSC 18mer-DHPE (0.05 to 5 pmol), were applied as 2 mm bands on a nitrocellulose coated FastSlide. Chondroitin sulphate C polysaccharide is a glycosaminoglycan (GAG). The presence of the carbohydrate-containing molecules on the support material was detected by an anti-chondroitin sulphate antibody, CS56. Binding of the antibody to the molecules was measured using the DAB colour reaction. The results of this experiment are shown in FIG. 8.

Similarly, different amounts of hyaluronic acid polysaccharide (HA) (5 to 500 ng) and its oligosaccharide derived NGL, HA 8mer-DHPE (0.05 to 5 pmol), were applied as 2 mm bands onto a nitrocellulose coated FastSlide. The interactions of TSG-6 (a protein which can bind to the HA) with the carbohydrate-containing molecules on the support material, HA and the HA NGL, are shown in FIG. 9.

As can be seen from FIG. 8, the sensitivity of detection of GAG-antibody binding was in the region of picomoles for the NGL and of nanograms for the CSC-polysaccharide. Therefore, a nitrocellulose coated slide can be used for detecting GAG-molecule binding.

A similar result can be seen in FIG. 9. Here the sensitivity of detection of hyaluronic acid polysaccharide (HA) was in the region of picomoles for the NGL and of nanograms for the HA polysaccharide. Therefore, a nitrocellulose coated slide can be used for detecting HA-molecule binding.

Binding of anti-Le^{x} Antibody to LNFP III as Aminopyridine and ADHP Derivatives on a Nitrocellulose-Coated FastSlide.

Different amounts of oligosaccharide derivatives were applied as 2 mm bands on a nitrocellulose coated FastSlide. The arrangement of the NGLs on the support and quantity used is shown under the data. The ADHP (bands 1 a-6a) and aminopyridine (PA) derivative (bands 3b-6b) of the Le^{x} pentasaccharide LNFP III were detected by an anti-Le^{x} antibody. The tetrasaccharide LNNT-ADHP was used as negative control (positions 1b and 2b). Binding of the antibody to the molecules was measured using the DAB colour reaction. The results of this experiment are shown in FIG. 10.

As can be seen in FIG. 10, the sensitivity of detection of the pentasaccharide LNFP III ADHP was in the region of picomoles. Therefore, a nitrocellulose coated slide can be used for detecting NGL-molecule binding, and the longer chain ADHP is the tag of choice.

### EXAMPLE 3

### Immobilisation of Neoglycolipids on Silica Gel Glass Plate

This example demonstrates the utility of a silica gel-coated slide as a support on which to immobilise reducing end-tagged carbohydrate molecules.

Different amounts of the fluorescent Lewis^{X} (Le^{x}) pentasaccharide neoglycolipid LNFP III-ADHP were applied on a silica gel-coated glass slide in 2 mm bands (1, 2, 5, 10, 35 and 70 pmol). After washing with PBS at room temperature for 3 hours, the retained neoglycolipid on the matrix was measured by its fluorescence and compared with freshly applied unwashed bands. The results of this are presented in FIG. 11. The fluorescence intensities were scanned and shown in the upper panel, and the percentage of neoglycolipid retained shown in the lower panel.

The data presented in FIG. 11 demonstrates that neoglycolipids can be immobilised on a silica gel-coated glass slide and are retained after washing at PBS. Therefore, silica gel-coated glass slide slides are a suitable support material on which to immobilise reducing end-tagged carbohydrate molecules.

### EXAMPLE 4

### Immobilisation of Neoglycolipids on Aluminium Oxide Gel Glass Slide

This example demonstrates the utility of a metal oxide gel-coated slide as a support on which to immobilise reducing end-tagged carbohydrate molecules.

Different amounts of the fluorescent neoglycolipid LNFP III-ADHP (as used in Example 2) were applied on an aluminium oxide gel-coated glass slide as 2 mm bands (1, 2, 5, 10, 35 and 70 pmol). After washing with PBS at room temperature for 3 hours, the retained neoglycolipid on the matrix was measured by its fluorescence and compared with freshly applied unwashed bands. The results of this are presented in FIG. 12. The fluorescence intensities were scanned and shown in the upper panel, and the percentage of neoglycolipid retained shown in the lower panel.

The data presented in FIG. 12 demonstrates that neoglycolipids can be immobilised on an aluminium oxide gel-coated glass slide and are retained after washing at PBS. Therefore, aluminium oxide gel-coated glass slides are a suitable support material on which to immobilise reducing end-tagged carbohydrate molecules.

### EXAMPLE 5

### A Method of Detecting a Molecule in a Test Sample

A test sample of body fluid is taken from the patient suspected of suffering from a disorder caused by a microbial infection.

A support according to the first aspect of the invention is prepared having immobilised a reducing end-tagged carbohydrate molecules to which a molecule indicative of the presence of a specific microbial infection will bind. For example, the molecule could be an antibody which recognises an epitope contained within the immobilised reducing end-tagged carbohydrate molecules.

The test sample is applied to the support and the binding of a molecule in the test sample to a reducing end-tagged carbohydrate molecule immobilised on the support is then measured using, for example, a fluorescence detection system. From the data generated it is possible to determine whether the patient is suffering from a microbial infection and, hence, benefit from treatment.

### EXAMPLE 6

### A Method to Identify Whether a Molecule Binds to a Reducing End-Tagged Carbohydrate Molecule

An array of reducing end-tagged carbohydrate molecules representing the carbohydrate composition of a specific cell type or tissue (in this example a human brain) is prepared on a support according to the first or second aspects of the invention. Hence the array is a complex mixture of reducing end-tagged carbohydrate molecules representing the glycome of a human brain. The reducing end-tagged carbohydrate molecules in this example are neoglycolipids.

The array is contacted with a quantity of a molecule, for example a possible therapeutic molecule, and any binding of a molecule in the test sample to a the array of neoglycolipids is measured using, for example, a fluorescence detection system. From the data generated it is possible to determine whether the possible therapeutic molecule interacts with a component of the glycome of the human brain. Such a method may be of particular use in screening possible therapeutic molecules to identify molecules which may be suitable for further analysis.

### EXAMPLE 7

### A Method of Measuring the Kinetics of Interaction Between a Molecule and a Reducing End-Tagged Carbohydrate Molecule

An array of reducing end-tagged carbohydrate molecules is prepared on a support according to the first aspect of the invention. The reducing end-tagged carbohydrate molecules arrayed are identical, however the quantity of reducing end-tagged carbohydrate molecules varies at different locations throughout the array.

The array is contacted with a quantity of a molecule known to interact with the arrayed reducing end-tagged carbohydrate molecule.

The kinetics of interaction of a molecule to any of the arrayed reducing end-tagged carbohydrate can be measured by real time changes in, for example, colorimetric or fluorescent signals.

Such a method may be of particular use in, for example, identifying a therapeutic molecule which can bind with a high affinity to reducing end-tagged carbohydrate molecules.

### EXAMPLE 8

### A Method to Identify a Reducing End-Tagged Carbohydrate Molecule in a Heterogeneous Population of Molecules

An array of reducing end-tagged carbohydrate molecules representing the carbohydrate composition of a specific cell type or tissue (in this example a human brain) is prepared on a support material according to the first aspect of the invention. Hence the array is a complex mixture of reducing end-tagged carbohydrate molecules representing the glycome of a human brain. The reducing end-tagged carbohydrate molecules in this example are neoglycolipids.

The array is contacted with a quantity of a heterogeneous population of molecules, for example a possible therapeutic molecules, and binding of any of the molecules to the array of neoglycolipids is measured using, for example, a fluorescence detection system. From the data generated it is possible to identify possible therapeutic molecules from a heterogeneous population of molecules which may be suitable for further analysis.

### EXAMPLE 9

### A Method to Identify a Carbohydrate Bound by a Molecule (Optionally from a Heterogeneous Population of Carbohydrates)

An array of reducing end-tagged carbohydrate molecules representing the carbohydrate composition of a specific cell type or tissue (in this example a human brain) is prepared on a support according to the first aspect of the invention. Hence the array is a complex mixture of reducing end-tagged carbohydrate molecules representing the glycome of a human brain. The reducing end-tagged carbohydrate molecules in this example are neoglycolipids.

The array is contacted with a quantity of a molecule, for example a possible therapeutic molecule, and binding of the molecule to the array of neoglycolipids is measured using, for example, a fluorescence detection system.

Should a complex mixture of neoglycolipids be identified as binding to the molecule, then the specific neoglycolipid in this population which binds to the molecule can be identified.

One method of identifying the specific neoglycolipid from a complex population of neoglycolipids is to use a deconvolution strategy. For example, a ‘daughter’ array of the complex mixture of neoglycolipids separated into individual or a restricted number of molecules can be generated. The molecule is then used to screen the daughter array and any neoglycolipids to which the molecule binds can be identified using mass spectrometry preceded as necessary by thin-layer or multi-dimentional chromatographies and chromatogram binding.

### EXAMPLE 10

### A Method of Separating Microbes or Specific Cells from a Heterogeneous Population of Microbes or Cells

A support according to the first aspect of the invention is prepared, on which is immobilised reducing end-tagged carbohydrate molecules is used to identify microbes, for example, from the urine of a patient having, or suspected of having, a urinary infection caused by a bacterium.

Further examples of this method of ‘panning’ for specific microbes cells in a hetereogeneous population will be obvious to a person skilled in the art. Also possible are methods for detecting and identifying other microbes, such as viruses or virally infected cells, using the same principle as that outlined above.

### EXAMPLE 11

### A Method to Identify Whether a Molecule Interferes with the Binding of a Molecule or Cell to a Reducing End-Tagged Carbohydrate Molecule

A support according to the first aspect of the invention is prepared having immobilised a homogenous population of reducing end-tagged carbohydrate molecules, in this case neoglycolipids. The support is then exposed to a quantity of molecules or cells which binds with the immobilised neoglycolipids.

Once prepared, the support having the neoglycolipid/molecule complex is then contacted with a quantity of a test molecule or cell, in this example a small drug. Any interference in the binding of the molecule or cell to the neoglycolipid by the small drug can be measured by, for example, detecting changes in the quantity of molecule binding to the neoglycolipid.

The method described above is a competition/inhibition assay which, as would be appreciated by a person skilled in the art, could be the basis for a screen to identify possible therapeutic molecules which affect the binding of, for example, a microbe to a carbohydrate-containing molecule present on a cell surface. The screen can be modified such that pools of test molecules can be screened to identify a test molecule which interferes with the binding of a molecule to a reducing end-tagged carbohydrate molecule from a heterogeneous population of test molecules, as will be appreciated by a person skilled in the art.

1-72 (canceled).

73. An array of reducing end-tagged carbohydrate molecules immobilized on a support.

74. An array as defined in claim 73 wherein the reducing end-tagged carbohydrate molecules are selected from the group consisting of glycolipids, neoglycolipids, carbohydrate molecules having a chromophore, oligosaccharides, monosaccharides and combinations thereof.

75. An array as defined in claim 74 wherein any neoglycolipids comprise a tag of between 24 to 50 carbon atom length.

76. An array as define in claim 73 wherein the reducing end-tagged carbohydrate molecules comprise a tag of between 5 to 25 carbon atoms with an aliphatic or aromatic hydrocarbon backbone.

77. An array as defined in claim 74 wherein any oligosaccharide is selected from the group consisting of N-glycans, O-glycans, O-glycans that terminate in N-acetylgalactosamine or N-acetylgalactosaminitol, O-glycans that terminate in mannose or mannitol, GPI-linked glycans, fragments of a glycosaminoglycan and combinations thereof.

78. An array as defined in either of claims 74 or 77 wherein any oligosaccharide or monosaccharide is derived from the group consisting of one or more carbohydrate sources selected from glycoproteins, glycolipids, proteoglycans/glycosaminoglycans and polysaccharides and chemically synthesized molecules.

79. An array as defined in claim 78 wherein the oligosaccharide or monosaccharide is a reducing sugar.

80. An array as defined in claim 78 wherein the oligosaccharide or monosaccharide is a reduced sugar.

81. An array as defined in claim 80 wherein the reduced oligosaccharide or monosaccharide is tagged at the reducing terminal after a mild oxidation procedure.

82. An array as defined in claim 73 wherein there are one or more samples of reducing end-tagged carbohydrate molecules which comprise a homogeneous sample of carbohydrate.

83. An array as defined in claim 73 wherein there are one or more samples of reducing end-tagged carbohydrate molecules which comprise a heterogeneous sample of carbohydrate.

84. An array as defined in claim 73 wherein the reducing end-tagged carbohydrate molecules comprise carbohydrate derived from a microbe.

85. An array as defined in claim 73 wherein the reducing end-tagged carbohydrate molecules comprise carbohydrate molecules from the group consisting of molecules derived from a specific cell type and molecules derived from a specific tissue or organ.

86. An array as defined in claim 85 where the cell type or tissue is derived from an animal.

87. An array as defined in claim 86 wherein the animal is a human.

88. An array as defined in claim 85 wherein the cell type or tissue is derived from a plant.

89. An array as defined in claim 73 wherein the support is selected from the group consisting of supports that are or include a hydrophobic membrane.

90. An array as defined in claim 89 where the hydrophobic membrane is one selected from the group consisting of membranes that are or comprise a nitrocellulose membrane, a PVDF membrane, and a nylon membrane.

91. An array as defined in claim 73 wherein the support comprises a material selected from the group consisting of a metal oxide and a metal oxide gel.

92. An array as defined in claim 91 wherein the metal oxide is aluminum oxide.

93. An array as defined in claim 73 wherein the support comprises a gel.

94. An array as defined in claim 93 wherein said gel is selected from the group consisting of a silica gel and an aluminum oxide gel.

95. A method of preparing an array of reducing end-tagged carbohydrate molecules immobilized on a support including the step of immobilizing the reducing end-tagged carbohydrate molecules on the support while solubilized in a solvent comprising an aqueous/aliphatic alcohol mixture.

96. A method as in claim 95 wherein said array includes molecules selected from the group including glycolipids, neoglycolipids, carbohydrate molecules having a chromophore, oligosaccharides, monosaccharides and combinations thereof.

97. A method as in either claim 95 or 96 wherein said solvent includes between 8 to 15% of an aliphatic alcohol selected from a list consisting of propanol (propan-1-ol), iso-propanol (propan-2-ol), n-butanol (butan-1-01), iso-butanol (butan-2-ol), and t-butanol (2-methylpropan-2-ol).

98. A method for detecting a molecule in a test sample or determining whether a molecule interacts with a carbohydrate comprising the steps of:
i) providing a test sample;
ii) contacting an array of reducing end-tagged carbohydrate molecules immobilized on a support with the test sample or molecule and performing a step selected from;
iii) detecting the binding of any molecules in the test sample to the array; or
iv) measuring whether a molecule of interest binds to any arrayed reducing end-tagged carbohydrate molecules.

99. A method as in claim 98 including the step of measuring the kinetics of interaction between a molecule of interest and an arrayed reducing end-tagged carbohydrate molecule.

100. A method of identifying a carbohydrate-binding or a carbohydrate bound by a molecule or molecules in a heterogeneous sample of molecules comprising:
i) contacting an array of reducing end-tagged carbohydrate molecules immobilized on a support with a sample selected from the group consisting of heterogeneous samples of molecules and samples of a molecule of interest; and performing a step selected from
ii) identifying a molecule or molecules which interact with the arrayed reducing end-tagged carbohydrate molecules; and
iii) identifying the reducing end-tagged carbohydrate molecule or molecules on the array to which the molecule of interest binds.

101. The method of claim 101 wherein step (iii) further comprises a deconvolution process.

102. A method of separating specific cells from a heterogeneous population of cells:
i) providing an array of reducing end-tagged carbohydrate molecules immobilized on a support, wherein the reducing end-tagged carbohydrate molecule or molecules is able to interact with specific cells;
ii) contacting the array with a heterogeneous population of cells; and
iii) separating those cells that bind to the array from those cells that do not bind to the array.

103. A method of determining whether a test molecule interferes with the binding of a molecule or cell to a reducing end-tagged carbohydrate molecule comprising:
i) providing an array of reducing end-tagged carbohydrate molecules immobilized on a support, wherein the reducing end-tagged carbohydrate molecule or molecules is bound by a molecule or cell;
ii) contacting the array with a test molecule;
iii) identifying whether the test molecule interferes with the binding of a molecule or cell to the reducing end-tagged carbohydrate molecule or molecules on the array.

104. The method of claim 103 wherein the test molecule is part of a heterogeneous population of molecules.

105. A method as defined in any one of claims 98, 100, 102 or 103 wherein the molecule or test molecule is a polypeptide.

106. A method as defined in claim 105 wherein the polypeptide is selected from the group consisting of antibodies, enzymes, receptors, lectins and glycoproteins.

107. A method as defined in any one of claims 98, 100, 102 or 103 wherein the molecule or test molecule is selected from the group consisting of peptidomimetics, nucleic acids, carbohydrates, lipids, glycolipids, hormones, microbial antigens, and glycomimics.

108. A method as defined in any one of claims 98, 100, 102 or 103 wherein the molecule or test molecule is a therapeutic molecule selected from the group consisting of a prophylactic-agent, a vaccine and an immunomodulator.

109. A method as defined in claim 108 wherein the therapeutic molecule is smaller than 500 daltons.

110. A method of preparing an array of reducing end-tagged carbohydrate molecules immobilized on a support including the step of using a solvent comprising an aliphatic alcohol for solubolizing said reducing end-tagged carbohydrate molecule.

111. A use as defined in claim 110 wherein the reducing end-tagged carbohydrate molecules are selected from the group consisting of glycolipids and neoglycolipids.

112. A method according to either claim 110 or 111 wherein the solvent includes between 8 to 15% of an aliphatic alcohol selected from the group consisting of propanol (propan-1-ol, iso-propanol (propan-2-ol), n-butanol (butan-1-ol), iso-butanol (butan-2-ol, and t-butanol (2-methylpropan-2-ol and combinations thereof.
